SEQUENCE LISTING (6 sequences)

SEQ ID NO: 1 - protein, 362 aa, Artificial Sequence
  recombinant P97 protein
MHHHHHHSSGLVPRGSGMKETAAAKFERQHMDSPDLGTDDDDKAMADIGSEFAPQTITELCSEYRNTQIYTINDKILSYTESMAGKREMVIITFKSGATFQVEVPGSQHIDSQKKAIERMKDTLRIAYLTETKIDKLCVWNNKTPNSIAAISLEGIPTKEGKREEVDKKVKELDNKIKGILPQPPAAKPEAAKPVAAKPEAAKPVAAKPEAAKPEAAKPVAAKPEAAKPVAAKPEAAKPVATNEGTPNQGKKAEGAPNQGKKAEGAPSQGKKAEGASNQQSPTTELTNYLPELGKKIDEIIKKQGKNWKTEVELIEDNIAGDAKLLYFVLRDDSKSGDPKKSSLKVKITVKQSNNNQELKSK

SEQ ID NO: 2 - protein, 234 aa, Artificial Sequence
  recombinant protein from PCV2
MTYPRRRFRRRRHRPRSHLGQILRRRPWLVHPRHRYRWRRKNGIFNTRLSRTIGYTVKKTTVRTPSWNVDMMRFNINDFLPPGGGSNPLTVPFEYYRIRKVKVEFWPCSPITQGDRGVGSTAVILDDNFVTKANALTYDPYVNYSSRHTITQPFSYHSRYFTPKPVLDRTIDYFQPNNKRNQLWLRLQTTGNVDHVGLGTAFENSIYDQGYNIRITMYVQFREFNLKDPPLNPK

SEQ ID NO: 3 - DNA, 36 bp, Artificial Sequence
  PCV2d ORF2 forward primer
cgcggatccgccgccaccatgacgtatccaaggagg

SEQ ID NO: 4 - DNA, 27 bp, Artificial Sequence
  PCV2d ORF2 reverse primer
ggcaagctttcatttagggttaagtgg

SEQ ID NO: 5 - DNA, 20 bp, Artificial Sequence
  PCV2 forward primer
aaccacatactggaaaccac

SEQ ID NO: 6 - DNA, 20 bp, Artificial Sequence
  PCV2 reverse primer
ttgaggagtaccattccaac

(19) United States
(12) Patent Application Publication
(10) Pub. No.: US 2024/0415950 A1
(43) Pub. Date: Dec. 19, 2024

(54) MULTIVALENT VACCINE COMPOSITION FOR PREVENTION OF PORCINE MYCOPLASMA AND PORCINE CIRCOVIRUS INFECTIONS

(71) Applicants: INNOVAC, Chuncheon-si (KR); KNU-INDUSTRY COOPERATION FOUNDATION, Chuncheon-si (KR)

(72) Inventors: Tae Wook HAHN, Chuncheon-si (KR); Ki Ju KIM, Chuncheon-si (KR); Woo Sung SHIN, Chuncheon-si (KR)

(73) Assignees: INNOVAC, Chuncheon-si (KR); KNU-INDUSTRY COOPERATION FOUNDATION, Chuncheon-si (KR)

(21) Appl. No.: 18/703,680

(22) PCT Filed: Mar. 4, 2022
(86) PCT No.: PCT/KR2022/003112

§ 371 Date: Apr. 22, 2024

(30) Foreign Application Priority Data
Mar. 4, 2021 (KR) 10-2021-0028980
Jun. 7, 2021 (KR) 10-2021-0073554

(51) Int. Cl. A61K 39/12; A61K 39/00; A61K 39/02; A61P 31/04; A61P 31/20; C12N 7/00

(52) U.S. Cl. CPC A61K 39/12; A61K 39/0241; A61P 31/04; A61P 31/20; C12N 7/00; A61K 2039/521; A61K 2039/545; A61K 2039/552; A61K 2039/55566; A61K 2039/575; A61K 2039/70; C12N 2750/10034

(57) ABSTRACT

The present invention relates to a multivalent vaccine composition for prevention of porcine mycoplasma and porcine circovirus infections and an infection prevention method using same. The multivalent vaccine composition according to the present invention exhibits a reciprocally supplemental effect on immune responses in pigs used as experimental animals and target animals and does not allow for interference between antigens. When inoculated into pigs, the vaccine composition induces high serological levels of antibody titers and neutralizing antibody titers and produces a high level of IFN-γ for Mhp or PCV2 stimulation in PBMC. Thus, the vaccine composition can find advantageous applications for preventing porcine mycoplasma and porcine circovirus infections.

### TECHNICAL FIELD

The present invention relates to a multivalent vaccine composition for prevention of porcine mycoplasma and porcine circovirus infections.

### BACKGROUND ART

Among various causes of infectious diseases that cause decreased production in pig farms, Mycoplasma hyopneumoniae (Mhp), Mycoplasma hyorhinis (Mhr), and Porcine circovirus type 2 (PCV2) are known to be the most dangerous cause. Mycoplasma hyopneumoniae (Mhp) is a respiratory pathogen associated with porcine respiratory disease complex (PRDC) and enzootic pneumonia (EP), and attracts attention as the causative agent of porcine mycoplasmal pneumonia by causing infection starting from colonization in respiratory ciliated cells. Mycoplasma hyopneumoniae alone shows symptoms such as dry cough when infected alone, but causes serious respiratory symptoms, reduces the activity of macrophages, and further increases the risk of infection with additional pathogens, including loss of the primary defense function to prevent invasion by other pathogens when combined with other respiratory pathogens.

It has been reported that Mycoplasma hyorhinis (Mhr) is emerging as a pathogen that plays a new role in respiratory infectious diseases in pigs following Mycoplasma hyopneumoniae, and further worsens the disease symptoms through mixed infection with porcine genital respiratory disease virus. In addition, it is known that Mycoplasma hyorhinis alone may cause mycoplasmic lesions expressed in Mycoplasma hyopneumoniae.

Meanwhile, porcine circovirus type 2 (PCV2) is a causative agent of postweaning multisystemic wasting syndrome (PMWS), and infected pigs becomechronic weight loss, have pale skin, and have an enlarged lymph nodes. In addition, the PCV2 shows respiratory symptoms such as difficulty breathing and chronic pneumonia and digestive symptoms such as diarrhea and stomach ulcers, and has a high mortality rate (to 50%) in weaning pigs, thereby causing high economic losses worldwide. The PCV2 is accompanied by many other infections, but is not sterilized by common disinfectants due to very strong resistance to the environment and organic compounds, and thus only vaccination is known to be the only alternative.

In particular, existing commercial vaccines were produced with PCV2a (or PCV2b) genotype, but since 2012, a genotype shift has occurred to the newly emerged PCV2d, and recently, at least 90% of PCV2d genotypes have been isolated. The PCV2a genotype is known to have nucleotide sequence homology of 91.4% with PCV2b, 90.4% with PCV2d, and the PCV2b genotype is known to have nucleotide sequence homology of 93.9% with PCV2d.

To prevent infection with Mycoplasma hyopneumoniae, Mycoplasma hyorhinis, or porcine circovirus type 2, which causes great damage to pig farms, a single or multivalent vaccine in an inactivated form has been currently commercialized and used. Particularly, for the prevention of porcine mycoplasma pneumonia, the inactivated vaccine is the only vaccine available. However, most commercialized Mycoplasma hyopneumoniae inactivated vaccines do not induce antibodies after inoculation, and have technical limitations in that the spread of natural infectious bacteria and colonization of respiratory cilia may not be prevented although clinical symptoms may be alleviated. In addition, in the case of multivalent vaccines, there is a problem that mixed individual antigens may interfere with each other to reduce efficacy.

In particular, there are various types of subspecies of Mycoplasma hyopneumoniae, Mycoplasma hyorhinis, and porcine circovirus type 2, which have a very high possibility of interference.

Accordingly, there is a need to develop a new multivalent vaccine so as to simultaneously prevent natural infections with porcine mycoplasma infections, especially Mycoplasma hyopneumoniae and Mycoplasma hyorhinis, and PCV2 including the existing prevalent PCV2b and the recently prevalent PCV2d and effectively form antibodies.

### DISCLOSURE

### Technical Problem

The present inventors developed a multivalent vaccine composition for prevention of porcine mycoplasma and porcine circovirus infections, confirmed that antibodies were produced by each antigen in an animal model inoculated with the multivalent vaccine composition, and then completed the present invention.

Therefore, an object of the present invention is to provide a multivalent vaccine composition for prevention of porcine mycoplasma and porcine circovirus infections.

Another object of the present invention is to provide a method for preventing porcine mycoplasma and porcine circovirus infections.

Yet another object of the present invention is to provide a pharmaceutical composition for preventing diseases caused by porcine mycoplasma and porcine circovirus infections.

### Technical Solution

An aspect of the present invention provides a multivalent vaccine composition for prevention of porcine mycoplasma and porcine circovirus infections, including (i) a porcine Mycoplasma hyopneumoniae (Mhp) strain; (ii) a porcine Mycoplasma hyorhinis (Mhr) strain; (iii) a porcine Mycoplasma hyopneumoniae-derived recombinant P97 protein; and (iv) a porcine circovirus type 2 (PCV2)-derived recombinant protein.

In addition, another aspect of the present invention provides a method for preventing porcine mycoplasma and porcine circovirus infections including inoculating the multivalent vaccine composition of the present invention into pigs.

In addition, yet another aspect of the present invention provides a pharmaceutical composition for preventing diseases caused by porcine mycoplasma and porcine circovirus infections, including (i) a porcine Mycoplasma hyopneumoniae (Mhp) strain; (ii) a porcine Mycoplasma hyorhinis (Mhr) strain; (iii) a porcine Mycoplasma hyopneumoniae-derived recombinant P97 protein; and (iv) a porcine circovirus type 2 (PCV2)-derived recombinant protein.

### Advantageous Effects

According to the present invention, a multivalent vaccine composition of Mhp+Mhr+recombinant P97 protein+recombinant PCV2 capsid antigen combination exhibits a reciprocally supplemental effect on immune responses in pigs used as experimental animals and target animals and does not allow for interference between antigens. When inoculated into pigs, the multivalent vaccine composition induces high serological levels of antibody titers and neutralizing antibody titers to each antigen and produces a high level of interferon-gamma (IFN-γ) for Mhp or PCV2 stimulation in peripheral blood mononuclear cells (PBMC), and when inoculated into pigs used as target animals and challenged, the multivalent vaccine composition shows excellent defense ability without side effects. Thus, the multivalent vaccine composition can find advantageous applications for preventing porcine mycoplasma and porcine circovirus infections.

### DESCRIPTION OF DRAWINGS

FIG. 1 illustrates results of confirming a recombinant PCV2d capsid protein according to the present invention using a transmission electron microscope.

FIG. 2 illustrates results of inoculating mice with the multivalent vaccine composition according to the present invention and confirming antibody titers against Mhp.

FIG. 3 illustrates results of inoculating mice with a multivalent vaccine composition according to the present invention and confirming antibody titers against recombinant P97 protein.

FIG. 4 illustrates results of inoculating mice with a multivalent vaccine composition according to the present invention and confirming antibody titers against Mhr.

FIG. 5 illustrates results of inoculating guinea pigs with a multivalent vaccine composition according to the present invention and a PCV2-alone vaccine and confirming antibody titers against a recombinant PCV2d capsid protein antigen.

FIG. 6 illustrates results of inoculating guinea pigs with a multivalent vaccine composition according to the present invention and a PCV2-alone vaccine and confirming neutralizing antibody titers against PCV2b.

FIG. 7 illustrates results of inoculating guinea pigs with a multivalent vaccine composition according to the present invention and a PCV2-alone vaccine and confirming neutralizing antibody titers against PCV2d.

FIG. 8 illustrates results of inoculating pigs with a multivalent vaccine composition according to the present invention and confirming antibody titers against Mhp.

FIG. 9 illustrates results of inoculating pigs with a multivalent vaccine composition according to the present invention and confirming antibody titers against recombinant P97 protein.

FIG. 10 illustrates results of inoculating pigs with a multivalent vaccine composition according to the present invention and confirming antibody titers against Mhr.

FIG. 11 illustrates results of inoculating pigs with a multivalent vaccine composition according to the present invention and confirming the secretion of interferon-gamma (IFN-γ) according to Mhp stimulation.

FIG. 12 illustrates results of inoculating pigs with a multivalent vaccine composition according to the present invention and confirming the secretion of IFN-γ according to PCV2 stimulation.

FIG. 13 illustrates the defense ability against challenge of the multivalent vaccine composition according to the present invention, and illustrates results of confirming body weight changes in pigs inoculated or noninoculated with the multivalent vaccine composition.

FIG. 14 illustrates the defense ability against challenge of the multivalent vaccine composition according to the present invention, and illustrates results of confirming average daily weight gain (ADWG) in pigs inoculated or noninoculated with the multivalent vaccine composition.

FIG. 15 illustrates the defense ability against challenge of the multivalent vaccine composition according to the present invention, and illustrates results of confirming PCV2b-specific IgG antibody titers in serum in pigs inoculated or noninoculated with the multivalent vaccine composition.

FIG. 16 illustrates the defense ability against challenge of the multivalent vaccine composition according to the present invention, and illustrates results of confirming PCV2d-specific IgG antibody titers in serum in pigs inoculated or noninoculated with the multivalent vaccine composition.

FIG. 17 illustrates the defense ability against challenge of the multivalent vaccine composition according to the present invention, and illustrates results of confirming PCV2b neutralizing antibody titers in pigs inoculated or noninoculated with the multivalent vaccine composition.

FIG. 18 illustrates the defense ability against challenge of the multivalent vaccine composition according to the present invention, and illustrates results of confirming PCV2d neutralizing antibody titers in pigs inoculated or noninoculated with the multivalent vaccine composition.

FIG. 19 illustrates the defense ability against challenge of the multivalent vaccine composition according to the present invention, and illustrates results of confirming IFN-γ secretion amounts in pigs inoculated or noninoculated with the multivalent vaccine composition.

FIG. 20 illustrates the defense ability against challenge of the multivalent vaccine composition according to the present invention, and illustrates results of quantifying the PCV2 DNA copy number of serum samples in each experimental group in pigs inoculated or noninoculated with the multivalent vaccine composition.

FIG. 21 illustrates the defense ability against challenge of the multivalent vaccine composition according to the present invention, and illustrates results of quantifying the PCV2 DNA copy number of nasal samples in each experimental group in pigs inoculated or noninoculated with the multivalent vaccine composition.

FIG. 22 illustrates the defense ability against challenge of the multivalent vaccine composition according to the present invention, and illustrates results of quantifying the PCV2 DNA copy number of rectal samples in each experimental group in pigs inoculated or noninoculated with the multivalent vaccine composition.

FIG. 23 illustrates the defense ability against challenge of the multivalent vaccine composition according to the present invention, and illustrates results of quantifying the PCV2 DNA copy number of lung samples in each experimental group in pigs inoculated or noninoculated with the multivalent vaccine composition.

FIG. 24 illustrates the defense ability against challenge of the multivalent vaccine composition according to the present invention, and illustrates results of quantifying the PCV2 DNA copy number of lymph node samples in each experimental group in pigs inoculated or noninoculated with the multivalent vaccine composition.

### BEST MODE OF THE INVENTION

Hereinafter, the present invention will be described in detail.

The present invention provides a multivalent vaccine composition for prevention of porcine mycoplasma and porcine circovirus infections, including (i) a porcine Mycoplasma hyopneumoniae (Mhp) strain: (ii) a porcine Mycoplasma hyorhinis (Mhr) strain: (iii) a porcine Mycoplasma hyopneumoniae-derived recombinant P97 protein; and (iv) a porcine circovirus type 2 (PCV2)-derived recombinant protein.

In the present invention, the ‘vaccine’ is a veterinary vaccine containing an antigenic substance and is administered for the purpose of inducing specific, active or passive immunity against Mhp, Mhr or PCV2.

Meanwhile, the infection with Mycoplasma hyopneumoniae begins when the pathogen adheres to the cilia of respiratory epithelial cells. Adhesin P97 is a membrane surface protein of Mycoplasma hyopneumoniae and is known as a highly immunogenic antigen. There are R1 and R2 regions consisting of repeated sections at the C terminus of P97, and the R1 region binds to an adhering site (AAKPV/E) of host respiratory cilia. The combined Mycoplasma hyopneumoniae may obtain amino acids required for growth from a host and may arrange the host's molecules variously to invade wounded tissue. Meanwhile, the host may produce antibodies to prevent Mycoplasma hyopneumoniae from adhering to pig cilia or to inhibit the growth of the adhering pathogen. However, according to previous studies, it has been reported that existing commercial vaccines may not induce antibodies against P97.

In this regard, the multivalent vaccine composition of the present invention may include a recombinant P97 protein to induce antibodies against P97. In the present invention, the recombinant P97 protein included in the vaccine composition may be prepared by fusing a porcine Mycoplasma hyopneumoniae-derived P97 gene and an E. coli heat-labile enterotoxin subunit B (eltb) gene. More specifically, the recombinant P97 protein may be prepared by fusing C-terminus repeat sequences R1 and RIR2 of the Mycoplasma hyopneumoniae P97 gene and the eltb of E. coli and amplifying P97 genes ltbR1 and ltbRIR2 fused with LTB by polymerase chain reaction (PCR). The recombinant P97 protein may be characterized as consisting of an amino acid sequence represented by the following SEQ ID NO: 1.

(SEQ ID NO: 1)
MHHHHHHSSGLVPRGSGMKETAAAKFERQHMDSPDLGTDDDDKAMADIGS
EFAPQTITELCSEYRNTQIYTINDKILSYTESMAGKREMVIITFKSGATF
QVEVPGSQHIDSQKKAIERMKDTLRIAYLTETKIDKLCVWNNKTPNSIAA
ISLEGIPTKEGKREEVDKKVKELDNKIKGILPQPPAAKPEAAKPVAAKPE
AAKPVAAKPEAAKPEAAKPVAAKPEAAKPVAAKPEAAKPVATNEGTPNQG
KKAEGAPNQGKKAEGAPSQGKKAEGASNQQSPTTELTNYLPELGKKIDEI
IKKQGKNWKTEVELIEDNIAGDAKLLYFVLRDDSKSGDPKKSSLKVKITV
KQSNNNQELKSK.

The recombinant P97 protein of the present invention may contain all polypeptides having at least 70%, 80%, 90%, 95%, 98% and preferably at least 99% homology with the amino acid sequence represented by SEQ ID NO: 1. The “homology” refers to a measure of similarity between proteins or polynucleotide sequences. These polypeptides may have deletion, addition or substitution of at least one amino acid compared to the amino acid sequence represented by SEQ ID NO: 1. The degree of homology between two sequences scored is based on the percentage of identity and/or preserving sequence substitutions.

In addition, the recombinant protein derived from porcine circovirus type 2 (PCV2) may be prepared as follows. Recombinant Bacmid DNA was prepared by inserting an ORF2 gene of PCV2d DNA isolated from pig serum into a pFastBac1 vector (Gibco, USA), and then transfected into an ExpiSf9 cell (Gibco) to prepare recombinant baculovirus. The PCV2-derived recombinant protein of the present invention may be characterized by consisting of an amino acid sequence represented by SEQ ID NO: 2.

(SEQ ID NO: 2)
MTYPRRRFRRRRHRPRSHLGQILRRRPWLVHPRHRYRWRRKNGIFNTRLS
RTIGYTVKKTTVRTPSWNVDMMRFNINDFLPPGGGSNPLTVPFEYYRIRK
VKVEFWPCSPITQGDRGVGSTAVILDDNFVTKANALTYDPYVNYSSRHTI
TQPFSYHSRYFTPKPVLDRTIDYFQPNNKRNQLWLRLQTTGNVDHVGLGT
AFENSIYDQGYNIRITMYVQFREFNLKDPPLNPK.

The PCV2-derived recombinant protein of the present invention may include all polypeptides having at least 70%, 80%, 90%, 95%, 98% and preferably at least 99% homology with the amino acid sequence represented by SEQ ID NO: 2. The “homology” refers to a measure of similarity between proteins or polynucleotide sequences. These polypeptides may have deletion, addition or substitution of at least one amino acid compared to the amino acid sequence represented by SEQ ID NO: 2. The degree of homology between two sequences scored is based on the percentage of identity and/or preserving sequence substitutions.

When the recombinant proteins of the present invention are used in the production of vaccines, the recombinant proteins may be added to the vaccine in the form of purified proteins or may be expressed in E. coli and included in an unpurified lysate state.

The Mycoplasma hyopneumoniae strain and the porcine Mycoplasma hyorhinis strain included in the multivalent vaccine composition according to the present invention are isolated from pigs with mycoplasmic lung lesions or known strains may be used without limitation, and each or all thereof may be characterized as inactivated, and for inactivation, methods known in the art may be used without limitation.

In the multivalent vaccine composition according to the present invention, Mycoplasma hyopneumoniae may be included in the vaccine at a concentration of 1×10⁸ to 1×10⁹ CCU (Colour-changing units)/mL, preferably 1.5×10⁸ to 5×10⁸ CCU/mL, and more preferably 2×10⁸ CCU/mL. At this time, it is preferable that the vaccine dose is 2 mL.

In the multivalent vaccine composition according to the present invention, Mycoplasma hyorhinis may be included in the vaccine at a concentration of 1×10⁸ to 1×10⁹ colour-changing units (CCU)/mL, preferably 1.5×10⁸ to 5×10⁸ CCU/mL, and more preferably 2×10⁸ CCU/mL. At this time, it is preferable that the vaccine dose is 2 mL.

In the multivalent vaccine composition according to the present invention, the porcine Mycoplasma hyopneumoniae-derived recombinant P97 protein may be included in the vaccine at a concentration of 10 to 100 μg/dose, preferably 20 to 50 μg/dose, and more preferably at least 25 μg/dose. At this time, it is preferable that the vaccine dose is 2 mL.

In the multivalent vaccine composition according to the present invention, the recombinant protein derived from porcine circovirus type 2 (PCV2) may be included in the vaccine at a concentration of 0.1 to 1000 μg/dose, preferably 10 to 30 μg/dose, and more preferably 20 μg/dose. At this time, it is preferable that the vaccine dose is 2 mL.

The multivalent vaccine composition of the present invention is an immunopotentiator capable of inducing protective immunity with minimal administration by enhancing the immunogenicity of the vaccine, and may include an adjuvant mixture and one or more pharmaceutically or veterinarily acceptable carriers, excipients or diluents. The “pharmaceutically or veterinarily acceptable” refers to a composition that is physiologically acceptable and does not generally cause an allergic reaction, such as gastrointestinal disorder, dizziness, etc., or a similar reaction thereto when administered to animals. Examples of the carrier, the excipient, and the diluent may include lactose, dextrose, sucrose, sorbitol, mannitol, xylitol, erythritol, maltitol, starch, acacia gum, alginate, gelatin, calcium phosphate, calcium silicate, cellulose, methyl cellulose, polyvinylpyrrolidone, water, methyl hydroxybenzoate, propyl hydroxybenzoate, talc, magnesium stearate, and mineral oil. In addition, the pharmaceutical composition may further include fillers, anti-coagulating agents, lubricants, wetting agents, flavorings, emulsifiers, preservatives and the like. Suitable carriers for use may include aqueous media including saline, phosphate buffered saline, a minimum essential medium (MEM), or an aqueous medium including MEM in HEPES buffer, but are not limited thereto. In an embodiment of the present invention, IMS1313, an IMS series excipient, was used considering safety and immune sustainability.

In addition, the multivalent vaccine composition of the present invention may include one or more adjuvants suitable for forming the vaccine composition.

The adjuvant that may be included in the composition of the present invention refers to a substance that enhances the immune responses of an injected animal, and a plurality of different adjuvants is known to those skilled in the art. The adjuvant includes Freunds complete and incomplete adjuvants, vitamin E, nonionic blocking polymers, muramyl dipeptide, Quil A, mineral oil and non-mineral oil, Carbopol, water-in-oil emulsion adjuvants, etc., but are limited thereto.

Further, the multivalent vaccine composition of the present invention may be formulated by using methods known in the art so as to provide rapid, sustained, or delayed release of the active ingredient after administrated to mammals. The formulation may be powders, granules, tablets, emulsions, syrups, aerosols, soft or hard gelatin capsules, sterile injectable solutions, and sterile powder forms. The multivalent vaccine composition of the present invention may be administered by intramuscular, subcutaneous, transdermal, intravenous, intranasal, intraperitoneal or oral routes, and preferably administered by intramuscular or subcutaneous routes. The dose of the vaccine may be appropriately selected depending on several factors such as the route of administration, age, sex, body weight, and severity of an animal, etc.

In addition, the multivalent vaccine composition of the present invention may be prepared by standard methods in the art, except for methods specific to the present invention, such as the production of the recombinant protein P97 and the PCV2-derived recombinant protein. For example, the organism may be grown in a culture medium, such as a complete medium, and the growth of the organism may be monitored by standard techniques such as measuring colour-changing units (CCU) and collected when sufficiently high titers are achieved. The stock may be further concentrated or lyophilized by conventional methods before included in the vaccine for formulation.

Further, the present invention provides a method for preventing porcine mycoplasma and porcine circovirus infections including inoculating the multivalent vaccine composition of the present invention into pigs.

The pigs may include, without limitation, subjects likely to be infected with mycoplasma and/or porcine circovirus, and such an infection prevention method may be used in combination with other treatment or prevention methods known in the art. As used in the present invention, the term “inoculation” means providing the composition of the present invention to a subject by any suitable method.

The multivalent vaccine composition according to the present invention may be inoculated once at 1 to 5 weeks of age, and more preferably once at 3 weeks of age.

The prevention method of the present invention is to prevent the occurrence of infectious diseases in pigs that may be caused by the porcine mycoplasma and/or porcine circovirus.

Further, the present invention provides a pharmaceutical composition for preventing diseases caused by porcine mycoplasma and porcine circovirus infections, including (i) a porcine Mycoplasma hyopneumoniae (Mhp) strain: (ii) a porcine Mycoplasma hyorhinis (Mhr) strain: (iii) a porcine Mycoplasma hyopneumoniae-derived recombinant P97 protein; and (iv) a porcine circovirus type 2 (PCV2)-derived recombinant protein.

The diseases caused by porcine mycoplasma and porcine circovirus infections may be one or more selected from the group consisting of porcine respiratory disease complex (PRDC), enzootic pneumonia (EP), postweaning multisystemic wasting syndrome (PMWS), porcine dermatitis and nephropathy syndrome (PDNS), sow abortion and mortality syndrome (SAMS), porcine reproductive and respiratory syndrome (PRRS), pseudorabies, Glasser's disease, streptococcal meningitis, salmonellosis, postweaning colibacillosis, dietetic hepatosis, suppurative bronchopneumonia, Eustachian tube inflammation, polyserositis, mycoplasma pneumonia and pleurisy pneumonia, but are not limited thereto.

The pharmaceutical composition of the present invention is preferably administered orally or parenterally.

In the case of oral administration, the oral administration includes intraoral administration, and the pharmaceutical composition of the present invention is not limited thereto, but may be orally administered in any orally acceptable form including pills, sugarcoated pills, capsules, solutions, gels, syrups, slurries, and suspensions.

In the case of oral tablets, commonly used carriers include lactose and corn starch. A lubricant such as magnesium stearate is also typically added. For oral administration in a capsule form, useful diluents include lactose and dried cornstarch. When aqueous suspensions are administered orally, the active ingredient is combined with emulsions and suspensions. If necessary, sweetening and/or flavoring and/or coloring agents may be added.

A pharmaceutical composition for intraoral administration may be prepared by mixing the active ingredient with a solid excipient and may be prepared in the form of granules for preparation in the form of tablets or sugarcoated pills. Suitable excipients may use sugar forms such as lactose, sucrose, mannitol and sorbitol, or starch from corn, wheat flour, rice, potato or other plants, cellulose such as methyl cellulose, hydroxypropylmethyl-cellulose or sodium carboxymethylcellulose, carbohydrates such as gums including arabic gum and tragacanth gum, or protein fillers such as gelatin and collagen. If necessary, disintegrants or solubilizers in each salt form such as cross-linked polyvinylpyrrolidone, agar and alginic acid or sodium alginate may be added.

As used in the present invention, the term “parenteral” includes subcutaneous, intradermal, intravenous, intramuscular, intraarticular, intrasynovial, intrasternal, intrathecal, intralesional and intracranial injection or infusion techniques.

The pharmaceutical composition of the present invention may be in the form of a sterile injectable preparation as a sterile injectable aqueous or oil suspension. This suspension may be formulated according to techniques known in the art using suitable dispersing or wetting agents (e.g., Tween 80) and suspending agents. The sterile injectable preparation may also be a sterile injectable solution or suspension (e.g., a solution in 1,3-butanediol) in a non-toxic parenterally acceptable diluent or solvent. Vehicles and solvents that may be acceptably used include mannitol, water, a Ringer's solution and an isotonic sodium chloride solution. In addition, sterile fixed oils are usually used as a solvent or suspending medium. For this purpose, any mild fixed oil may also be used by including synthetic mono- or diglycerides. Fatty acids such as oleic acid and its glyceride derivatives are useful in injectable preparations, such as pharmaceutically acceptable natural oils (e.g., olive oil or castor oil), especially polyoxyethylated oils thereof. Further, in the case of parenteral administration, the pharmaceutical composition of the present invention may be prepared as an aqueous solution. Preferably, a physically appropriate buffer solution such as Hank's solution, Ringer's solution or physically buffered saline may be used. The aqueous injection suspension may be added with a substrate capable of increasing the viscosity of the suspension, such as sodium carboxymethylcellulose, sorbitol or dextran. In addition, the suspensions of the active ingredient may be prepared as suitable oily injection suspensions. Suitable lipophilic solvents or carriers include fatty acids such as sesame oil or synthetic fatty acid esters such as ethyl oleate, triglycerides or liposomes. Polycationic non-lipid amino polymers may also be used as carriers. Optionally, the suspension may use suitable stabilizers or agents to increase the solubility of the compound and to prepare a highly concentrated solution.

The pharmaceutical composition of the present invention may also be administered in the form of a suppository for rectal administration. These compositions may be prepared by mixing suitable non-irritating excipients that are solid at room temperature but liquid at rectal temperature with a mixture obtained by mixing (i) the porcine Mycoplasma hyopneumoniae (Mhp) strain: (ii) the porcine Mycoplasma hyorhinis (Mhr) strain: (iii) the porcine Mycoplasma hyopneumoniae-derived recombinant P97 protein; and (iv) the porcine circovirus type 2 (PCV2)-derived recombinant protein. These substances include cocoa butter, beeswax, and polyethylene glycol, but are not limited thereto.

When the pharmaceutical composition of the present invention is applied topically to the skin, the pharmaceutical composition should be formulated in a suitable ointment containing the active ingredient suspended or dissolved in the carrier. For topically administering the mixture obtained by mixing (i) the porcine Mycoplasma hyopneumoniae (Mhp) strain: (ii) the porcine Mycoplasma hyorhinis (Mhr) strain: (iii) the porcine Mycoplasma hyopneumoniae-derived recombinant P97 protein; and (iv) the porcine circovirus type 2 (PCV2)-derived recombinant protein of the present invention, the carrier includes mineral oil, liquid paraffin, white petrolatum, propylene glycol, polyoxyethylene, polyoxypropylene compounds, emulsifying wax, and water, but is not limited thereto. In addition, the pharmaceutical composition of the present invention may be formulated as a suitable lotion or cream containing the active compound suspended or dissolved in the carrier. Suitable carriers include mineral oil, sorbitan monostearate, polysorbate 60, cetyl ester wax, cetearyl alcohol, 2-octyldodecanol, benzyl alcohol, and water, but are not limited thereto. The pharmaceutical composition of the present invention may also be topically applied to the lower intestinal tract by rectal suppositories and also with suitable enemas. Topically applied transdermal patches are also included in the present invention.

The pharmaceutical composition of the present invention may be administered by intranasal aerosol or inhalation. Such a composition is prepared according to techniques well known in the field of medicine and may be prepared as a solution in saline using benzyl alcohol or other suitable preservatives, absorption enhancers for increasing bioavailability, fluorocarbons and/or other solubilizers or dispersants known in the art.

The specific effective amount for a specific subject may vary according to various factors including the activity of the mixture obtained by mixing (i) the porcine Mycoplasma hyopneumoniae (Mhp) strain: (ii) the porcine Mycoplasma hyorhinis (Mhr) strain: (iii) the porcine Mycoplasma hyopneumoniae-derived recombinant P97 protein; and (iv) the porcine circovirus type 2 (PCV2)-derived recombinant protein of the present invention, age, body weight, general health, sex, diet, administration time, administration route, excretion rate, drug combination, and the severity of a specific disease to be prevented or treated.

The above-described contents of the present invention are equally applied to each other unless otherwise contradict each other, and those appropriately modified and implemented by those skilled in the art are also included in the scope of the present invention.

Hereinafter, the present invention will be described in detail through Examples, but the scope of the present invention is not limited only to the Examples below.

### Example 1. Preparation of Antigen of Multivalent Vaccine Composition

1-1. Preparation of Mycoplasma hyopneumoniae Inactivated Antigen

Mycoplasma hyopneumoniae (M. hyopneumoniae, hereinafter Mhp) and Mycoplasma hyorhinis (hereinafter Mhr) strains were isolated from lungs showing mycoplasmic lung lesions in pigs, each strain was identified using PCR.

The isolated Mhp and Mhr strains were cultured for 3 days at 5% CO₂ and 37° C. using a Friis' medium. Through protein pattern analysis and random amplification of polymorphic DNA (RAPD) analysis, it was confirmed that the isolated strain was Mhp or Mhr. The strain solution cultured for 3 days had the bacterial count of approximately 0.1 to 5.0×10⁹ CCU/mL. The pH of the cultured strain solution was adjusted to about 7.8, and then the strains were inactivated for 20 to 24 hours at a final concentration of 4 mM using an inactivating reagent such as binary ethyleneimine (BEI). The BEI was prepared by adding L-bromoethylaminehydrobromide (BEA) (Sigma-Aldrich, St. Louis, MO, USA) to the culture medium. Thereafter, a neutralizing reagent, sodium thiosulfate (Sigma-Aldrich, St. Louis, MO, USA), was mixed to a final concentration of 16 mM, neutralized for 20 to 24 hours, and a portion of the inactivated culture medium was added to a new medium and then cultured at 37° C. for at least a week to confirm whether to be inactivated. The inactivated culture medium was concentrated by centrifugation and used as a vaccine antigen.

### 1-2. Preparation of Recombinant Mhp P97 Protein Antigen

A recombinant P97 protein was prepared by fusing an eltb gene to a P97 gene of Mycoplasma hyopneumoniae and then using a pET-30a (+) (Novagen, USA) expression system. In summary, E. coli BL21 codon-plus RIL competent cells (Novagen) were transformed with a recombinant plasmid using heat shock and then cultured in 1 L of a LB (Luria-Bertani) medium supplemented with 50 μg/mL kanamycin. Thereafter, the cells were mixed with 1 mM isopropyl-B-D-1-thiogalactopyranoside (IPTG) to induce protein expression for 20 hours, and then lysed and centrifuged to obtain a lysate containing the recombinant protein from the supernatant.

The obtained protein antigen was purified by the following two methods. First, the lysate containing the recombinant protein was bound to an Ni-NTA column using affinity and then washed at 5 CV (5-fold column resin volume) with a purification buffer (50 mM NaH₂PO₄, 300 mM NaCl) to remove the unbound protein. Thereafter, 5CV of non-specifically bound protein was removed with a purification buffer containing 40 mM imidazole, and the recombinant P97 protein antigen was eluted and recovered with the purification buffer containing 400 mM imidazole. The recovered protein was filtered and concentrated according to a volume. The purified protein antigen was quantified using a Bicinchoninic acid (BCA) method, and the purity was confirmed using a sodium dodecyl sulphate-polyacrylamide gel electrophoresis (SDS-PAGE) method.

Second, the pH of the lysate containing the recombinant protein was adjusted to 6.0 and stirred and reacted for about 2 hours. Thereafter, the protein antigen was precipitated by centrifugation, and the protein was recovered by adding phosphate buffer saline (PBS) at pH 7.2. The purified protein was quantified by the BCA method, and the purity was confirmed by the SDS-PAGE method.

The amino acid sequence of the recombinant P97 protein purified as above was represented in SEQ ID NO: 1.

### 1-3. Preparation of Recombinant PCV2d Capsid Protein Antigen

### 1-3-1. Preparation of Cells and Strains

PCV-free PK-15 cells were cultured in a Dulbecco's modified Eagle medium (DMEM) (GenDEPOT, Katy, TX, USA) added with 10% fetal bovine serum (FBS) (GenDEPOT)) and a 1% antibiotic-antimycotic solution (Gibco) under conditions of 37° C. and 5% CO₂. Insect cells (ExpiSf9, Gibco) used for protein expression were shaking-cultured under conditions of 28° C. and 100 rpm using an ExpiSf CD medium (Gibco).

An ORF2 base sequence of a PCV2d genotype was amplified using a viral DNA of PCV2d isolated from a pig farm located in Nonsan-si, Chungcheongnam-do in 2017 as a template, and a virus neutralization test for PCV2d was performed using an isolating and subculturing PCV2d from the lymph nodes and lung tissues of pigs determined to be infected with PCV2.

### 1-3-2. Preparation of Recombinant Baculovirus

Recombinant baculovirus was prepared using a Baculovirus Expression System (Gibco) according to the user instructions recommended by a manufacturer. In summary, an ORF2 gene of PCV2d was amplified using a forward primer (5′-CGCGGATCCGCCGCCCACCATGACGTATCCAAGGAGG-3′ (SEQ ID NO: 3)) and a reverse primer (5′-GGCAAGCTTTCATTTAGGGTTAAGTGG-3′ (SEQ ID NO: 4)) with TOPsimple DyeMIX-Forte (Enzynomics Inc., Daejeon, Korea). The amplified PCV2 capsid (PCV2 CP) gene was cleaved with restriction enzymes BamHI and HindIII (Enzynomics Inc.), purified using a HiYield Gel/PCR DNA Extraction Kit (RBC Bioscience, Taipei, Taiwan), and then cloned into a pFastBac1 (pFB) (Gibco) baculovirus vector. The recombinant pFB-2dCP was inserted into competent Escherichia coli DH10Bac™ (Gibco) and transformed, and sequencing was analyzed. Recombinant bacmid DNA was transfected into ExpiSf9 cells using an ExpiFectamine reagent (Gibco). The cells were shaking-cultured under conditions of 28° C. and 100 rpm for 5 days to obtain a recombinant baculovirus (Bac-2dCP) expressing PCV2d ORF2. Viral DNA was extracted using Ribospin™ vRD (GeneAll), and then the titer of the recombinant baculovirus was measured using real-time quantitative polymerase chain reaction (qPCR), which showed 1.48×10¹¹ copies/mL.

### 1-3-3. Purification of Expressed Recombinant Proteins

ExpiSf9 cells were infected with recombinant baculovirus at a concentration of 5 multiplicity of infection (MOI) and then cultured. On day 5 of culture, cells were centrifuged at 1,000×g for 5 minutes and harvested. The expressed recombinant protein was purified using an anion exchange chromatography column. Briefly, the harvested cells were resuspended in a lysis buffer containing 50 mM sodium dihydrogen phosphate dihydrate (NaH₂PO₄·2H₂O), 300 mM sodium chloride (NaCl), and 1% IGEPAL CA-630 (Sigma-Aldrich, St. Louis, MO, USA). The cells were reacted on ice for 30 minutes and then centrifuged at 10,000×g for 10 minutes. The supernatant containing the recombinant protein was purified on a Q Sepharose Fast Flow (GE Healthcare, Madison, WI, USA) ion exchange platform and then filtered through a 0.22 μm filter. The amino acid sequence of the PCV2-derived recombinant protein purified as above was represented by SEQ ID NO: 2.

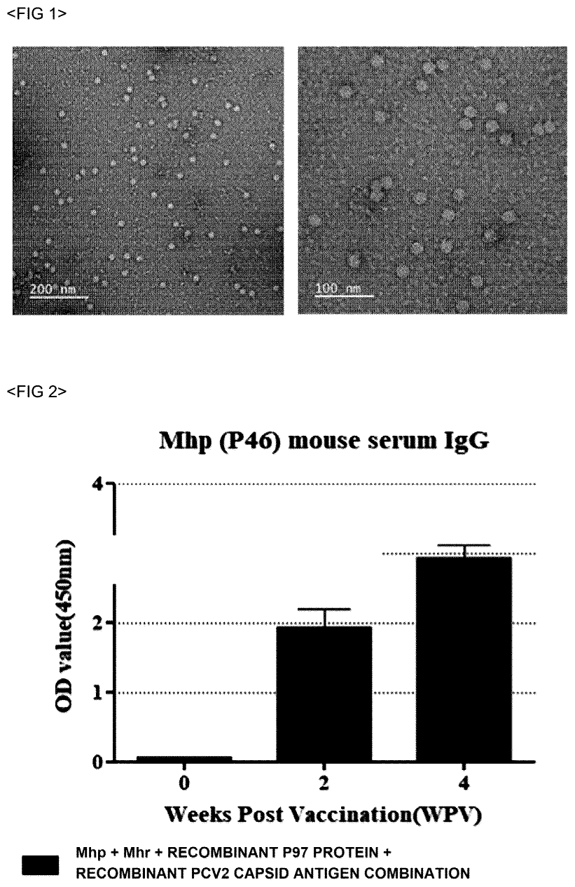

In addition, the PCV2-derived recombinant protein was observed using a transmission electron microscope (TEM: JEM-2100F: JEOL, Tokyo, Japan) at the Chuncheon Center of the Korea Institute for Basic Science, and as a result, it was confirmed that as illustrated in FIG. 1, PCV2 virus-like particles (VLP) with a diameter of about 17 nm were formed.

### 1-3-4. Recombinant Protein Expression Analysis

The purified recombinant protein was loaded into a 12% gel, subjected to SDS-PAGE, and then transferred to a hybond polyvinylpyrrolidone membrane (Amersham Biosciences, Freiburg, Germany) using a semidry transfer apparatus (Bio-Rad, Hercules, CA, USA). As a result of performing western blotting using porcine anti-PCV2 antibody (1:500 dilution) and horseradish peroxidase (HRP)-conjugated goat anti-porcine IgG antibody (1:5,000 dilution) (Bethyl Laboratories, Montgomery, TX, USA), protein bands of about 28 kDa were confirmed.

### 1-4. Preparation of Multivalent Vaccine Composition According to the Present Invention

The Mhp and Mhr inactivated strain antigens prepared through Example 1-1 at a concentration of 2×10⁸ CCU/dose or more, the recombinant P97 protein antigen prepared through Example 1-2 at a concentration of 25 μg/dose or more, and the recombinant PCV2d capsid protein antigen prepared in Example 1-3 at a concentration of 20 μg/dose were mixed in 2×PBS (1 mL/dose) and then stirred and mixed for 20 minutes or more. Then, the same amount of adjuvant (IMS1313, Seppic) was added and stirred for 20 minutes or more to prepare a multivalent vaccine composition.

### Example 2. Preparation of Experimental Animals

### 2-1. Preparation of Mice

Five BALB/c female mice with body weight of 15 g were inoculated with the multivalent vaccine composition (Mhp+Mhr+recombinant P97 protein+recombinant PCV2 capsid protein combination) according to the present invention. The multivalent vaccine according to the present invention (200 μL/dose, 1/10 of the pig dose) was inoculated into the thigh muscles of both hind legs of the mouse. Blood samples were collected at 0, 2, 4, and 6 weeks after inoculation and stored at −70° C. for serological analysis and then used in the experiment.

### 2-2. Preparation of Guinea Pigs

Four guinea pigs with body weight of 300 to 350 g were inoculated with the multivalent vaccine composition (Mhp+Mhr+recombinant P97 protein+recombinant PCV2 capsid protein combination) according to the present invention. In addition, four guinea pigs were inoculated with a PCV2-alone vaccine as a control group. Each vaccine (1 mL/dose, 1/2 of the pig dose) was inoculated into the thigh muscle of the hind leg of guinea pig in each experimental group. Blood samples were collected at 0, 2, 4, and 6 weeks after inoculation and stored at −70° C. for serological analysis and then used in the experiment.

### 2-3. Preparation of Pigs

Five pigs with body weight of 5 to 8 Kg were inoculated with the multivalent vaccine composition (Mhp+Mhr+recombinant P97 protein+recombinant PCV2 capsid protein combination) according to the present invention. The vaccine (2 mL/dose) according to the present invention was inoculated into the neck muscle of each pig. Blood samples were collected at 0, 4, 9, 14, and 17 weeks after inoculation and stored at −70° C. for serological analysis and then used in the experiment. In addition, for cytokine analysis, blood was collected from the jugular vein at 0, 4, 9, and 14 weeks after inoculation, peripheral blood mononuclear cells (PBMC) were isolated, and the formed IFN-γ was analyzed by stimulation with antigen.

### Example 3. Effect Testing Method of Multivalent Vaccine Composition According to the Present Invention

### 3-1. Enzyme-Linked Immunosorbent Assay (ELISA)

Indirect ELISA was performed for serological analysis following vaccination with according to inoculation of the multivalent vaccine composition according to the present invention. More specifically, specific IgG antibody titers against Mhp (identified in mice (recombinant P46 protein)), recombinant P97 protein (identified in pigs and mice), and PCV2 (identified in guinea pigs and pigs) were examined. Briefly, the recombinant P97 protein (100 ng/well), the recombinant P46 protein (125 ng/well), or the recombinant PCV2 capsid protein (100 ng/well) was diluted to respective concentrations in a 0.05 M carbonate buffer (pH 9.6), and then 100 μL/well each was dispensed into a 96-well microplate (Nunc Maxisorp, Roskilde, Denmark) and coated at 4° C. for 16 hours. Each well was washed three times with phosphate-buffered saline (PBS)-T (0.05% Tween 20), added with 1% BSA-containing PBS and then reacted at 37° C. for 2 hours. Next, the cells were washed three times with PBS-T, and 100 μL of serum diluted to 1:100 was dispensed and reacted at 37° C. for 2 hours. After washing three times with PBS-T, the plate was dispensed with HRP-conjugated goat anti-mouse (or anti-guinea pig or anti-pig) IgG Antibody (1:10,000 dilution: Bethyl Laboratories) and reacted at 37° C. for 1 hour. After washed three times with PBS-T, each well was dispensed with a 3,3,5,5′-tetramethylbenzidine (TMB) substrate (TMB One Component HRP Microwell Substrate (Surmodics, Inc., Eden Prairie, MN, USA)) and reacted in the dark for 5 minutes, and the reaction was stopped with 2 N H₂SO₄. The optical density (OD) of each plate was measured at a wavelength of 450 nm using an Epoch microplate reader (BioTek, Winooski, VT, USA).

In addition, for serological analysis according to inoculation of the multivalent vaccine according to the present invention, sandwich ELISA was performed to examine IgG antibody titers specific to the Mhr P37 protein (identified in mice and pigs). Anti-P37 IgG (capture antibody) was diluted to a concentration of 500 ng/ml in a carbonate buffer, dispensed at 100 L/well into each well of a 96-well microplate, and then coated at 4° C. for 16 hours. The coating solution was removed, washed three times with PBS-T, and each well was dispensed with 200 μL of PBS-T containing 10% FBS as a blocking solution and reacted at 37° C. for 4 hours. The blocking solution was removed and washed three times with 0.05% PBST. Thereafter, the recombinant P37 protein was diluted to 500 ng/ml in the blocking solution, and then 100 μL each was dispensed into each well, and the recombinant P37 protein reacted with anti-P37 IgG for 1 hour at 37° C. Meanwhile, a test serum was prepared by diluting mouse or pig serum 1/100 in a 10% FBS solution (with 100 ng of an E. coli lysate) and reacting at 37° C. for 1 hour. The recombinant P37 protein solution was removed and washed three times with 0.05% PBS-T. 90 μL of the test serum was dispensed into each well of the plate reacted with the recombinant P37 protein and then reacted at 37° C. for 1 hour. The test serum was removed and washed three times with 0.05% PBS-T. An anti-mouse (or anti-pig) IgG HRP conjugate was diluted 1/20,000 in a 10% FBS solution and 100 μL each was added to each well. After reacting at 37° C. for 1 hour, the solution was removed and washed four times with 0.05% PBS-T. Thereafter, 100 μL of the TMB solution was added to each well and reacted in the dark for 5 minutes at room temperature. 100 μL of a stop solution was dispensed into each well and the absorbance was immediately measured at 450 nm.

In addition, for serological analysis according to inoculation of the multivalent vaccine according to the present invention, the IgG antibody titers specific to Mhp (identified in pigs) were examined using an MH kit from IDEXX.

### 3-2. Virus Neutralization Test (VN Test)

PCV2 neutralizing antibodies were measured using immunofluorescence assay (IFA). After vaccination, guinea pig or pig serum at 0, 2, 4, and 6 weeks was inactivated by heating at 56° C. for 30 minutes, and then binary serially diluted in DMEM, and PK-15 cells at a density of 70 to 80% were inoculated with an equal volume of PCV2b or PCV2d virus (200×50% tissue culture infectious dose, TCID₅₀) and cultured at 37° C. in 5% CO₂. After 72 hours, the infected cells were fixed with 90% acetone and added and reacted with fluorescein isothiocyanate (FITC)-labeled anti-guinea pig (or anti-pig) IgG (Bethyl Laboratories). Neutralizing antibody titers were determined as the highest serum dilution rate showing 90% or more of virus neutralization under a fluorescence microscope.

### 3-3. Measurement of Interferon-gamma (IFN-γ)

To evaluate the cell-mediated immune responses to the multivalent vaccine composition according to the present invention in pigs, the amount of IFN-γ secretion was examined. PBMCs isolated from blood at 0, 4, 9, and 14 weeks after inoculation with the multivalent vaccine composition according to the present invention were stimulated with Mhp inactivated or PCV2 antigen and reacted for 72 hours. The supernatant was separated and the amount of IFN-γ secretion was measured using a porcine IFN-γ ELISA kit (Invitrogen, Lofer, Austria) according to the manufacturer's instructions.

### 3-4. Statistical Analysis

Data were analyzed using GraphPad Prism 7 software (Graph-Pad Software, Inc., La Jolla, CA, USA), and each group showed a significant difference compared with a control group using one-way analysis of variance (ANOVA) with Dunnett's multiple comparison analysis. A P value of less than 0.05 was considered statistically significant.

### Example 4. Confirmation of Antibody Titers after Inoculation into Mice

### 4-1. Confirmation of Formation of Antibody Against Mhp

Mice, which were porcine mycoplasma vaccine test animals, were inoculated with the multivalent vaccine composition according to the present invention, and blood was collected at weeks 0, 2, and 4 to confirm antibody titers against Mhp. For this purpose, the IgG antibody titers specific to the recombinant P46 protein, an immunodominant protein known to be encoded by the Mhp genome, were measured. As a result, as illustrated in FIG. 2, even though the multivalent vaccine according to the present invention contained all of Mhp, Mhr, the recombinant P97 protein, and the recombinant PCV2 capsid protein, antibody titers were normally formed against Mhp, and antibody titers increased depending on the week of vaccination. Therefore, it was confirmed that interference by various antigens did not occur when inoculated the multivalent vaccine of the present invention.

### 4-2. Confirmation of Antibody Titers Against Mhp Recombinant P97 Protein

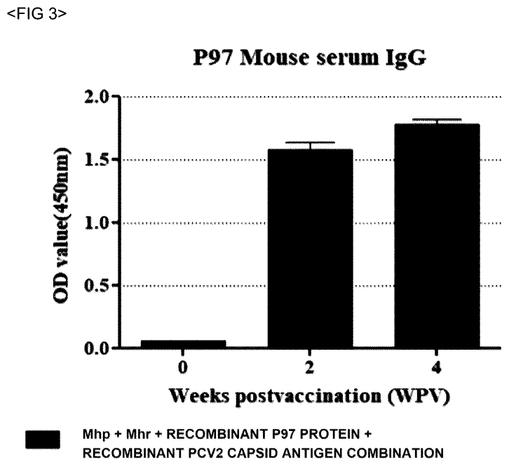

Mice, which were porcine mycoplasma vaccine test animals, were inoculated with the multivalent vaccine composition according to the present invention, and blood was collected at weeks 0, 2, and 4 to confirm antibody titers against the recombinant P97 protein. As a result, as illustrated in FIG. 3, similarly to the previous results, antibody titers against the recombinant P97 protein were formed normally, and antibody production rapidly increased in week 2 and high antibody titers were showed even in week 4.

### 4-3. Confirmation of Formation of Antibody Against Mhr

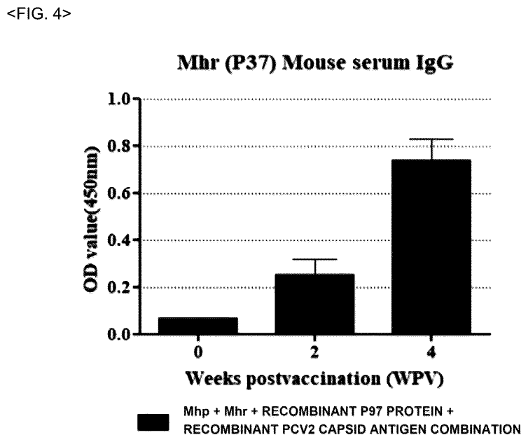

Mice, which were porcine mycoplasma vaccine test animals, were inoculated with the multivalent vaccine composition according to the present invention, and blood was collected at weeks 0, 2, and 4 to confirm antibody titers against Mhr. For this purpose, IgG antibody titers specific to the recombinant P37 protein known to be encoded by the Mhr genome were measured. As a result, as illustrated in FIG. 4, it was confirmed that the antibody titers against Mhr were formed normally and increased according to the week of vaccination.

Through the results above, it was confirmed that the multivalent vaccine composition of the Mhp+Mhr+recombinant P97 protein+recombinant PCV2 capsid protein combination of the present invention did not show mutual interference in mice, which were porcine mycoplasma vaccine test animals, and effectively formed antibodies against each antigen.

### Example 5. Confirmation of Antibody Titers after Inoculation into Guinea Pigs

### 5-1. Confirmation of Antibody Titers Against Recombinant PCV2 Capsid Protein Antigen

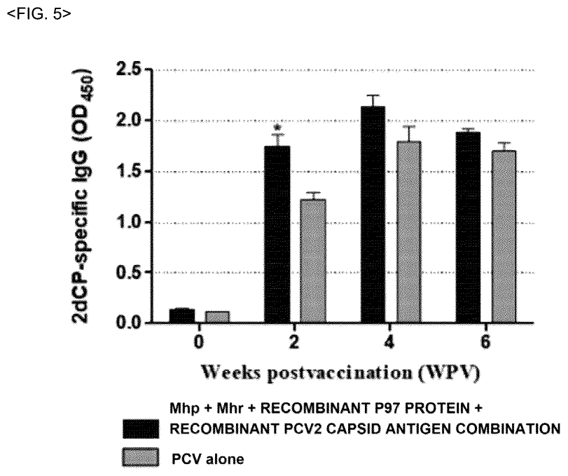

Guinea pigs were inoculated with the multivalent vaccine composition according to the present invention or PCV2-alone vaccine, and blood was collected at weeks 0, 2, 4, and 6, and antibody titers against the recombinant PCV2 capsid protein antigen were confirmed. As a result, as illustrated in FIG. 5, the multivalent vaccine composition according to the present invention showed significantly higher levels of IgG antibody titers than a PCV2-alone vaccination group at 2 weeks after vaccination. At week 4 after vaccination, all experimental groups showed the highest peak values, and the experimental group inoculated with the multivalent vaccine according to the present invention showed a higher antibody titer during the entire period. Therefore, it was confirmed that antibody titers against the recombinant PCV2 capsid protein antigen were generated more effectively when inoculated with the multivalent vaccine composition according to the present invention than with the PCV2-alone vaccine.

### 5-2. Confirmation of Neutralizing Antibody Titers Against PCV2b or PCV2d

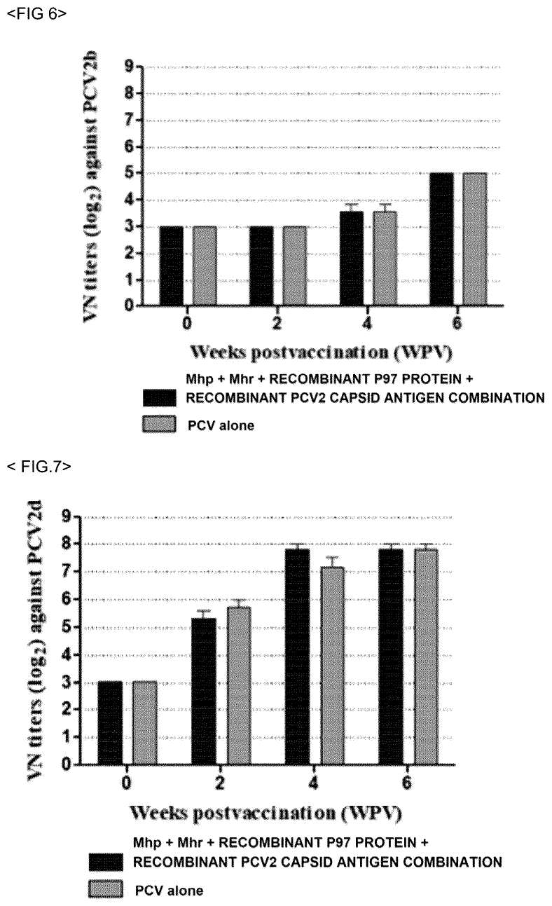

Guinea pigs were inoculated with the multivalent vaccine composition according to the present invention or PCV2-alone vaccine, and blood was collected at weeks 0, 2, 4, and 6, and neutralizing antibody titers against PCV2b or PCV2d genotype were confirmed and illustrated in FIGS. 6 and 7. As a result, it was confirmed that compared to the PCV2-alone vaccination group, the multivalent vaccine composition according to the present invention induced equal or slightly higher neutralizing antibody titers against the PCV2b or PCV2d genotype in the entire period after vaccination. In particular, it was confirmed that the antibody titers against PCV2d were higher than those against PCV2b.

### Example 6. Confirmation of Antibody Titers after Inoculation in Pigs

### 6-1. Confirmation of Antibody Titers to Mhp

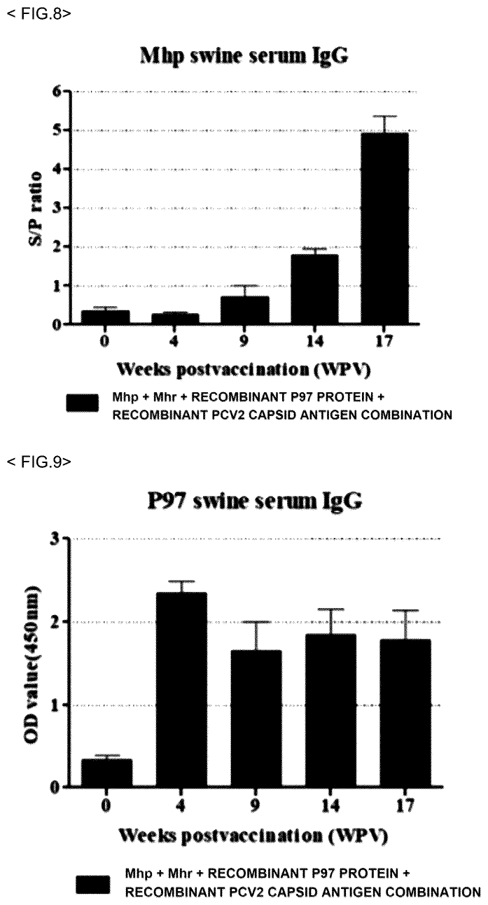

Pigs were inoculated with the multivalent vaccine composition according to the present invention, and blood was collected at weeks 0, 4, 9, 14, and 17 to confirm antibody titers against Mhp. As a result, as illustrated in FIG. 8, it was confirmed that the multivalent vaccine composition according to the present invention induced very high antibody titers up to 17 weeks, starting with antibody titers increased from 9 weeks after vaccination.

### 6-2. Confirmation of Antibody Titers Against Mhp Recombinant P97

Pigs were inoculated with the multivalent vaccine composition according to the present invention, and blood was collected at weeks 0, 4, 9, 14, and 17 to confirm antibody titers against the recombinant P97 protein. As a result, as illustrated in FIG. 9, it was confirmed that antibody titers were formed by showing a very high level of OD value only 4 weeks after vaccination. In particular, it was confirmed that interference with antibody titers against the P97 antigen was not shown by adding the PCV2 antigen.

### 6-3. Confirmation of Antibody Against Mhr

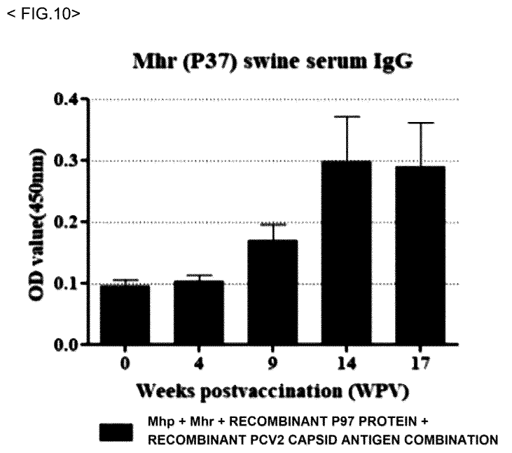

Pigs were inoculated with the multivalent vaccine composition according to the present invention, and blood was collected at weeks 0, 4, 9, 14, and 17 to confirm antibody titers against the recombinant P37 protein, and the formation of antibodies against Mhr was confirmed. As a result, as illustrated in FIG. 10, it was confirmed that antibodies against a Mhr-derived recombinant P37 protein were excellently formed when inoculated with the multivalent vaccine composition according to the present invention.

### 6-4. Confirmation of Interferon-Gamma (IFN-Gamma, IFN-γ) Secretion According to Mhp Stimulation

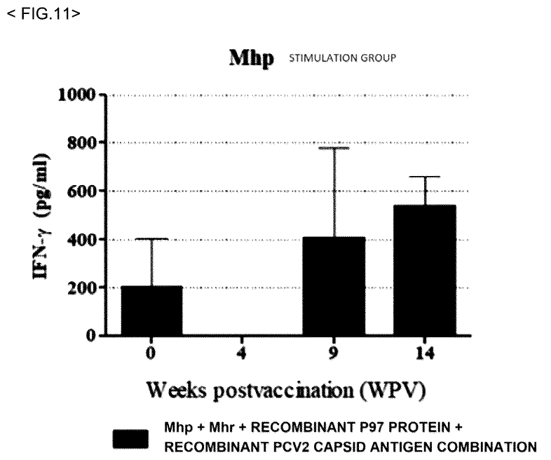

Pigs were inoculated with the multivalent vaccine composition according to the present invention, and blood was collected at weeks 0, 4, 9, and 14 to confirm IFN-y secretion according to Mhp stimulation. As a result, as illustrated in FIG. 11, it was confirmed that the amount of IFN-γ secretion increased from week 9 after vaccination and steadily increased until week 14.

### 6-5. Confirmation of IFN-γ Secretion According to PCV2 Stimulation

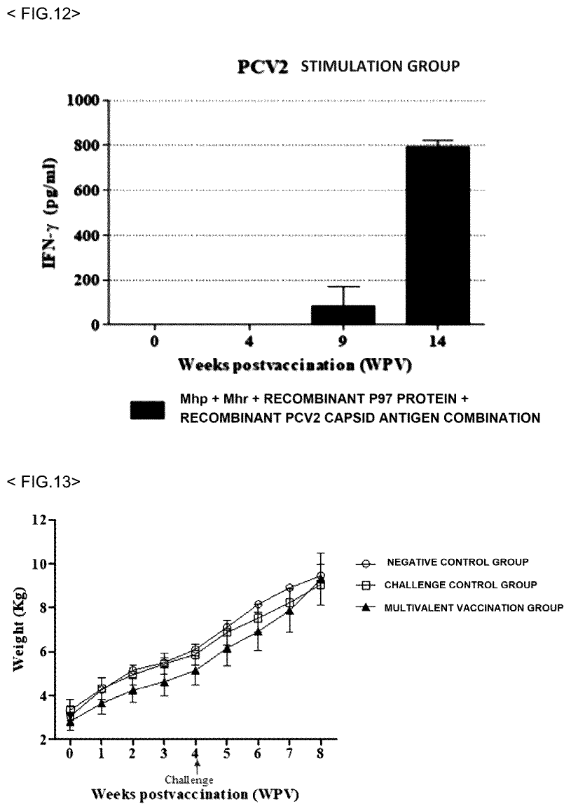

Pigs were inoculated with the multivalent vaccine composition according to the present invention, and blood was collected at weeks 0, 4, 9, and 14 to confirm IFN-y secretion according to PCV2 stimulation. As a result, as illustrated in FIG. 12, the amount of IFN-γ secretion increased from week 9 after vaccination and tended to increase steadily until week 14. In particular, a high level of IFN-γ was observed 14 weeks after vaccination.

### Example 7. Safety Evaluation of Multivalent Vaccine Composition According to the Present Invention

### 7-1. Inoculation of Multivalent Vaccine Composition in Pigs

In order to evaluate the safety and defense ability of the multivalent vaccine composition according to the present invention, an experimental group was set for specific pathogen-free (SPF) miniature pigs under the following conditions.

TABLE 1

| Experimental | | | Number | Inocu- |
|---|---|---|---|---|
| group | Description of group | | of | lation |
| name | Vaccination | Challenge | subjects | dose |
| Negative control | Non- | Not | 2 | — |
| group | vaccination | performed | | |
| Challenge control | Non- | | 3 | — |
| group | vaccination | | | |
| Group inoculated | Inoculated with | Performed | 5 | 1 dose |
| with multivaient | multivalent | | | (2 mL) |
| vaccine composition | vaccine | | | |
| according to present | composition | | | |
| invention | | | | |

When piglets were 3 weeks old, blood was collected and then vaccinated (week 0 after vaccination). In addition, blood was collected at 5 weeks of age (2 weeks after vaccination), 7 weeks of age (4 weeks after vaccination), 9 weeks of age (6 weeks after vaccination), and 11 weeks of age (8 weeks after vaccination). In addition, at 7 weeks of age, challenge was performed after blood collection, and at 11 weeks of age, euthanasia was performed to identify lesions in lymph nodes and lungs. As the challenge, 2 mL of a retained PCV2d isolate (HID9071, (titer: 10^{5.5} TCID₅₀/mL)) was inoculated intranasally.

The inoculation dose and the inoculation route were set with reference to the national lot release test standards, and more specifically, the multivalent vaccine composition according to the present invention was inoculated once (2 mL/dose) into the right neck muscle of the piglet. For the challenge, 2 mL of the PCV2d isolate was injected at 1 mL each through both nostrils. For blood collection, 5 mL of blood was collected through the jugular vein at 0 and 2 weeks postvaccination (WPV) to separate serum, and at 4, 6, 7, and 8 WPV, 8 mL of blood was collected through the jugular vein to separate PBMCs and serum.

### 7-2. Measurement of Body Weight

The body weights of each experimental group were measured every week during the test period. Using the measured body weights, the average daily weight gain (ADWG) for each subject was analyzed using Equation 1 below.

Average daily weight gain (ADWG)=(body weight in measurement week−body weight in previous week)/7 days [Equation 1]

As the result, as illustrated in FIG. 13 and Table 2 below, all groups before vaccination (0 WPV) showed an average body weight of between 2.82 and 3.33 Kg, and the group inoculated with the multivalent vaccine composition according to the present invention (hereinafter referred to as the multivalent vaccination group) at 4 WPV had an average weight of 5.14 Kg, slightly lower than the challenge control group (5.87 Kg) and the negative control group (6.10 Kg). However, the body weight at 4 weeks (8 WPV) after the challenge was an average of 9.05 Kg, which was the lowest in the challenge control group, and the multivalent vaccination group showed a similar level of body weight compared to the negative control group with an average of 9.29 Kg.

TABLE 2
Body weight for each group (Kg, AVE ± SE)

| | Negative | Challenge | Multivalent vaccination group |
|---|---|---|---|
| WPV | control group | control group | according to the present invention |
| 0 | 3.05 ± 0.05 | 3.33 ± 0.48 | 2.82 ± 0.43 |
| 1 | 4.25 ± 0.05 | 4.30 ± 0.50 | 3.64 ± 0.48 |
| 2 | 5.15 ± 0.05 | 4.93 ± 0.44 | 4.24 ± 0.57 |
| 3 | 5.50 ± 0.20 | 5.43 ± 0.48 | 4.62 ± 0.63 |
| 4 | 6.10 ± 0.10 | 5.87 ± 0.47 | 5.14 ± 0.67 |
| 5 | 7.10 ± 0.10 | 6.87 ± 0.55 | 6.15 ± 0.79 |
| 6 | 8.15 ± 0.05 | 7.52 ± 0.65 | 6.91 ± 0.88 |
| 7 | 6.90 ± 0.10 | 8.22 ± 0.17 | 7.87 ± 1.01 |
| 8 | 9.45 ± 0.05 | 9.05 ± 0.93 | 9.29 ± 1.18 |

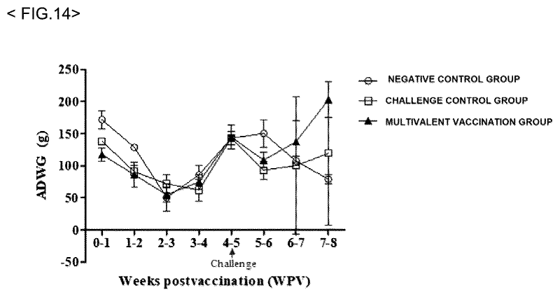

As the ADWG measured result, as illustrated in FIG. 14 and Table 3 below, from the vaccination to challenge (0 to 4 WPV), the average ADWG of the multivalent vaccination group was 82.86 g, which showed no significant difference compared to the challenge control group (90.48 g) and the negative control group (108.93 g). However, from the challenge until the end of the test (5 to 8 WPV), the challenge control group had the lowest average ADWG of 113.69 g, and the average ADWG of the multivalent vaccination group was 148.21 g, which was higher than the negative control group (119.64 g). Therefore, it was confirmed that there were no side effects such as lack of appetite (decreased appetite) due to inoculation of the multivalent vaccine composition according to the present invention.

TABLE 3

| | ADWG For each group (g, AVE ± SE) | | |
|---|---|---|---|
| | | | Multivalent vaccination |
| | Negative | Challenge | group according to the |
| WPV | control group | control group | present invention |
| 0-1 | 171.43 ± 14.29 | 138.10 ± 4.76 | 117.14 ± 10.50 |
| 1-2 | 128.57 ± 0.00 | 90.48 ± 9.52 | 85.71 ± 19.69 |
| 2-3 | 50.00 ± 21.43 | 71.43 ± 14.29 | 54.29 ± 11.43 |
| 3-4 | 85.71 ± 14.29 | 61.90 ± 16.67 | 74.29 ± 11.43 |
| 0-4 AVE | 108.93 | 90.48 | 82.86 |
| 4-8 | 142.86 ± 0.00 | 142.86 ± 10.91 | 144.29 ± 18.82 |
| 5-6 | 150.00 ± 21.43 | 92.86 ± 14.29 | 108.57 ± 12.25 |
| 6-7 | 107.14 ± 7.14 | 100.00 ± 107.22 | 137.14 ± 32.29 |
| 7-8 | 78.57 ± 7.14 | 119.05 ± 111.60 | 202.86 ± 27.90 |
| 4-8 AVE | 119.64 | 113.69 | 148.21 |
| 0-8 AVE | 114.29 | 102.08 | 115.54 |

### 7-3. Confirmation of Occurrence of Clinical Symptoms or Side Effects

Rectal body temperature was measured at 0, 4, 24, and 48 hours after inoculation with the multivalent vaccine composition according to the present invention. In addition, side effects at the injection site and clinical side effects were observed for 7 days.

The clinical side effects included loss of appetite, decreased vitality, cough, difficulty breathing, asphyxiation, shock, vomiting, and diarrhea.

As a result of body temperature measurement, as illustrated in Table 4 below, it was confirmed that the body temperature was slightly increased 4 hours after inoculation with the multivalent vaccine composition according to the present invention, but decreased to the pre-inoculation level after 24 and 48 hours.

TABLE 4

| | Group-specific body temperature | | | |
|---|---|---|---|---|
| | according to time elapsed | | | |
| Experimental | after vaccination (° C., AVE ± SE) | | | |
| group name | 0 hour | 4 hours | 24 hours | 48 hours |
| Negative control | 39.00 ± 0.35 | 39.50 ± 0.10 | 39.20 ± 0.05 | 39.00 ± 0.05 |
| group | | | | |
| Challenge control | 39.20 ± 0.09 | 39.60 ± 0.07 | 39.00 ± 0.09 | 39.40 ± 0.22 |
| group | | | | |
| Multivalent | 39.40 ± 0.09 | 40.80 ± 0.18 | 39.20 ± 0.07 | 38.90 ± 0.26 |
| vaccination group | | | | |
| according to the | | | | |
| present invention | | | | |

In addition, as a result of confirming clinical symptoms, the multivalent vaccination group according to the present invention did not show any clinical symptoms due to vaccination. Therefore, it was confirmed that the multivalent vaccine composition according to the present invention had no effect on body temperature and clinical symptoms.

### Example 8. Evaluation of Defense Ability of Multivalent Vaccine Composition of the Present Invention

### 8-1. Immunological Evaluation

### 8-1-1. Confirmation of PCV2 Antibody Titers

PCV2b-specific IgG and PCV2d-specific IgG antibody titers in the serum of each experimental group were confirmed using an indirect immunofluorescence assay (hereinafter, IFA). At this time, for each experimental group, the experiment was performed by collecting the serum at the time of vaccination or 2, 4, 6, and 8 weeks after vaccination.

First, 2×10⁴ cells/100 μL/well of PK-15 cells, which were PCV1-free porcine kidney cells, and the retained PCV2b (HID9029) or PCV2d (HID9071) virus were diluted to 600 TCID₅₀/mL, and then each 100 μL/well was dispensed into a 96-well plate.

The cells were cultured in an incubator of 37° C. and 5% CO₂ for 24 hours and then added with 200 μL/well of 300 mM glucosamine and reacted for 30 minutes to remove the supernatant. After washed three times with PBS, 200 μL/well of DMEM added with a 1% antibiotic-antimycotic solution (Gibco) was dispensed and cultured for 4 days. The supernatant was removed and washed once using PBS. 100 μL/well of 80% acetone was dispensed and fixed at 4° C. for 1 hour.

The fixing solution was discarded, washed once with PBS, and the plate was stored at −20° C. and used for an antibody titer test.

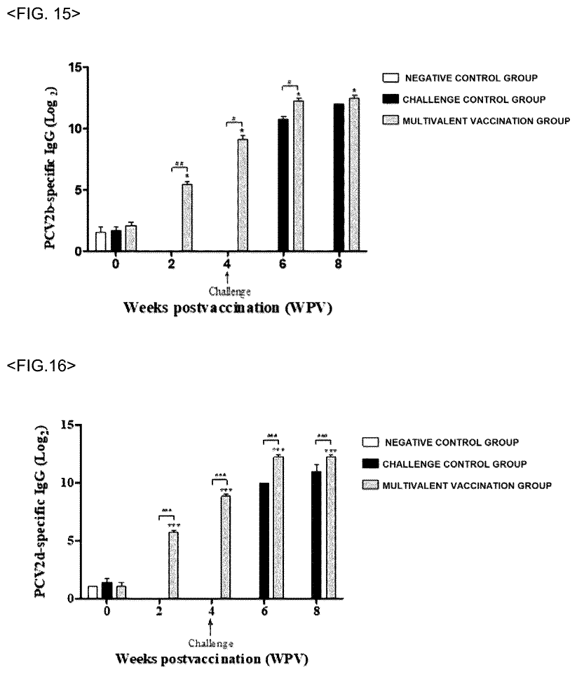

Pig serum samples were prepared by binary dilution with DMEM, dispensed at 100 μL/well on the fixed plate, and reacted at 37° C. for 1 hour. Thereafter, the samples were washed three times using PBS. The plate was dispensed with 100 μL/well of Pig IgG-heavy and light chain antibody (Goat polyclonal, FITC conjugate [Bethyl]) diluted 1:200 in PBS, and reacted at 37° C. for 30 minutes. Thereafter, the plate was washed once with PBS, observed under a fluorescence microscope, and the reciprocal of the highest dilution rate at which the virus was proliferated was determined as antibody titers. The results of PCV2b-specific IgG antibody titers confirmed above were shown in Table 5 and FIG. 15 below. In FIG. 15, * represented a result compared to a negative control group (P<0.05), #represented a result compared to a challenge control group (P<0.05), and ##represented a result compared to the challenge control group (P<0.01).

As a result, before vaccination (0 WPV), all experimental groups showed low levels of antibody titers. It was confirmed that the multivalent vaccination group according to the present invention induced PCV2b-specific IgG antibody titers significantly (P<0.01) compared to other experimental groups from 2 WPV. In particular, antibody titers continued to increase even after the challenge, and then significantly high levels of IgG antibody titers were shown until the end of the test.

The challenge control group induced antibody titers from 6 WPV, but showed a significantly (P<0.05) lower level than that of the multivalent vaccination group according to the present invention, and a negative control group had low levels of antibody titers throughout the entire test period.

TABLE 5

| Experimental | PCV2b-specific IgG average antibody titer (dilution rate, AVE ± SE) | | | | |
|---|---|---|---|---|---|
| group name | 0 WPV | 2 WPV | 4 WPV | 6 WPV | 8 WPV |
| Negative | 3.00 ± 1.00 | <2.00 ± 0.00 | <2.00 ± 0.00 | <2.00 ± 0.00 | <2.00 ± 0.00 |
| control group | | | | | |
| Challenge | 3.33 ± 0.67 | <2.00 ± 0.00 | <2.00 ± 0.00 | 1,706.67 ± 341.33 | 4,096.00 ± 0.00 |
| control group | | | | | |
| Multivalent | 4.40 ± 0.98 | 44.80 ± 7.84 | 563.20 ± 125.41 | 4,915.20 ± 819.20 | 5,734.40 ± 1,003.31 |
| vaccination group | | | | | |
| according to | | | | | |
| the present invention | | | | | |

In addition, the results of confirming the PCV2d-specific IgG antibody titers were shown in FIG. 16 and Table 6 below. At this time, in FIG. 16, *** represented a result compared with the negative control group (P<0.001), and ## #represented a result compared with the challenge control group (P<0.001).

As a result, before vaccination (0 WPV), all experimental groups showed low levels of antibody titers. It was confirmed that the multivalent vaccination group according to the present invention induced PCV2d-specific IgG antibody titers significantly (P<0.001) compared to other experimental groups from 2 WPV. In particular, IgG antibody titers continued to increase even after challenge, and then showed significantly high levels of IgG antibody titers until the end of the test.

The challenge control group induced antibody titers from 6 WPV, but showed significantly (P<0.001) lower levels than the multivalent vaccination group according to the present invention, and the negative control group showed low levels of antibody titers throughout the entire test period.

TABLE 6

| Experimental | PCV2d-specific IgG average antibody titer (dilution rate, AVE ± SE) | | | | |
|---|---|---|---|---|---|
| group name | 0 WPV | 2 WPV | 4 WPV | 6 WPV | 8 WPV |
| Negative | 2.00 ± 0.00 | <2.00 ± 0.00 | <2.00 ± 0.00 | <2.00 ± 0.00 | <2.00 ± 0.00 |
| control group | | | | | |
| Challenge | 2.67 ± 0.67 | <2.00 ± 0.00 | <2.00 ± 0.00 | 1,024.00 ± 0.00 | 2,048.00 ± 1,024.00 |
| control group | | | | | |
| Multivalent | 2.40 ± 0.40 | 51.20 ± 7.84 | 460.80 ± 51.20 | 4,915.20 ± 819.20 | 4,915.20 ± 819.20 |
| vaccination group | | | | | |
| according to | | | | | |
| the present invention | | | | | |

### 8-1-2. Confirmation of PCV2 Neutralizing Antibody Titers

Neutralizing antibody titers in the serum of each experimental group were confirmed as follows. First, the serum of each experimental group was inactivated at 56° C. for 30 minutes. 50 μL of the serum and 150 μL of DMEM were dispensed into the first well of a 96-well plate and diluted, and then 100 μL/well of DMEM was dispensed into the remaining wells and binarily diluted. After diluting the retained PCV2b (HID9029) or PCV2d (HID9071) virus to 400 TCID₅₀/mL, an equal amount (100 μL/well) was dispensed into wells containing binary diluted serum and reacted at 37° C. for 1 hour. PK-15 cells, PCV1-free porcine kidney cells, were dispensed into each well at 2×10⁴ cells/100 μL/well. The cells were cultured in an incubator at 37° C. and 5% CO₂ for 24 hours, and then treated with 300 mM glucosamine at 200 μL/well for 30 minutes. Then, the supernatant was removed and washed three times using PBS. Thereafter, the cells were added with 200 μL/well of DMEM containing 1% antibiotic-antimycoticsolution and cultured for 2 days. After culturing, immunofluorescence assay was performed as follows.

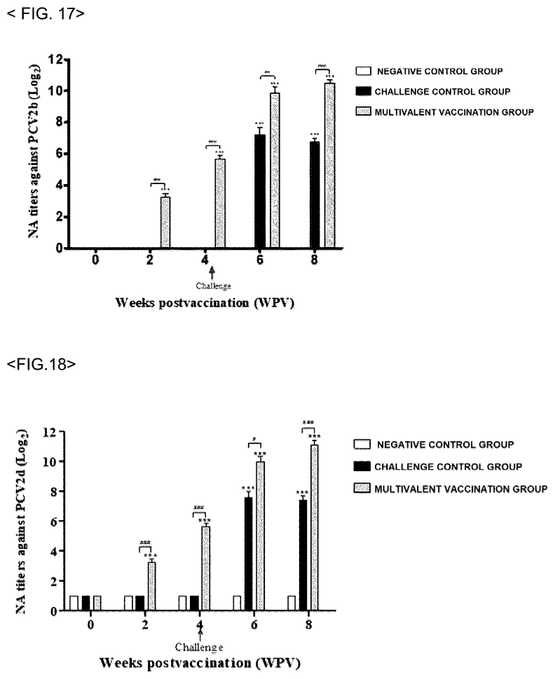

The culture medium was discarded and washed once with PBS, and the plate was placed in an incubator at 37° C. and dried for 30 minutes. 100 μL/well of 80% acetone was dispensed to the dried plate and then fixed at 4° C. for 1 hour. Then, the fixing solution was removed and washed once using PBS. Pig anti-PV2 antiserum (anti-PCV2 antiserum) diluted 1:500 in PBS was dispensed at 100 μL/well and reacted at 37° C. for 1 hour. Thereafter, the serum was washed three times using PBS. The plate was dispensed with 100 μL/well of Pig IgG-heavy and light chain antibody (Goat polyclonal, FITC conjugate [Bethyl]) diluted 1:200 in PBS, and reacted at 37° C. for 30 minutes. Thereafter, the plate was washed once using PBS and observed by a fluorescence microscope. At this time, the reciprocal of the highest serum dilution rate that inhibited virus proliferation (90% inhibition in 1 well) was determined as neutralizing antibody titers. The results of measuring the PCV2b neutralizing antibody titers confirmed above were shown in FIG. 17 and Table 7 below. In FIG. 17, *** represented a result compared to a negative control group (P<0.001), ##represented a result compared to a challenge control group (P<0.01), and ## #represented a result compared to the challenge control group (P<0.001).

As a result, before vaccination (0 WPV), all experimental groups showed low levels of neutralizing antibody titers. The multivalent vaccination group according to the present invention showed the average neutralizing antibody titer of 9.6 or more against PCV2b from 2 WPV, and thus it could be seen that neutralizing antibody titers against PCV2b began to be produced. In addition, at 6 WPV, high neutralizing antibody titers of average 921.6 or more were shown. In particular, neutralizing antibody titers continued to increase from 4 WPV after the challenge, and at 8 WPV, the high level of neutralizing antibody titer of average 1,433.6 or more was shown.

In the challenge control group, neutralizing antibody titers were produced after challenge, but at 8 WPV, compared to the multivalent vaccination group, low levels of neutralizing antibody titers were shown as the average 106.67 or more, and the negative control group showed very low levels of neutralizing antibody titers in the entire test period.

TABLE 7

| Experimental | PCV2b average neutralizing antibody titer (dilution rate, AVE ± SE) | | | | |
|---|---|---|---|---|---|
| group name | 0 WPV | 2 WPV | 4 WPV | 6 WPV | 8 WPV |
| Negative | 2.00 ± 0.00 | 2.00 ± 0.00 | 2.00 ± 0.00 | 2.00 ± 0.00 | 2.00 ± 0.00 |
| control group | | | | | |
| Challenge | 2.00 ± 0.00 | 2.00 ± 0.00 | 2.00 ± 0.00 | 149.33 ± 56.44 | 106.67 ± 21.33 |
| control group | | | | | |
| Multivalent | 2.00 ± 0.00 | 9.60 ± 1.00 | 51.20 ± 7.84 | 921.60 ± 298.54 | 1,433.60 ± 25.83 |
| vaccination group | | | | | |
| according to | | | | | |
| the present invention | | | | | |

Further, the results of measuring the PCV2d neutralizing antibody titers were shown in FIG. 18 and Table 8 below. At this time, in FIG. 18, *** represented a result compared to a negative control group (P<0.001), #represented a result compared to a challenge control group (P<0.05), and ## #represented a result compared to the challenge control group (P<0.001).

As a result, before vaccination (0 WPV), all experimental groups showed low levels of neutralizing antibody titers. The multivalent vaccination group showed the average neutralizing antibody titer of 9.6 or more against PCV2b from 2 WPV, and thus it could be seen that neutralizing antibody titers against PCV2b began to be produced. In addition, at 6 WPV, high neutralizing antibody titers increased to average 1,024 or more were shown. In particular, the neutralizing antibody titers continued to increase from 4 WPV after the challenge, and at 8 WPV, the very high level of neutralizing antibody titer of average 2,252 or more was shown.

In the challenge control group, neutralizing antibody titers were produced after challenge, but at the end of the test, compared to the multivalent vaccination group, low levels of neutralizing antibody titers were shown as, the average 170.67 or more, and the negative control group showed very low levels of neutralizing antibody titers in the entire test period.

TABLE 8

| Experimental | PCV2d average neutralizing antibody titer (dilution rate, AVE ± SE) | | | | |
|---|---|---|---|---|---|
| group name | 0 WPV | 2 WPV | 4 WPV | 6 WPV | 8 WPV |
| Negative control group | 2.00 ± 0.00 | 2.00 ± 0.00 | 2.00 ± 0.00 | 2.00 ± 0.00 | 2.00 ± 0.00 |
| Challenge control group | 2.00 ± 0.00 | 2.00 ± 0.00 | 2.00 ± 0.00 | 192.00 ± 64.00 | 170.67 ± 42.67 |
| Multivalent vaccination | 2.00 ± 0.00 | 9.60 ± 1.60 | 51.20 ± 7.84 | 1,024.00 ± 280.43 | 2,252.80 ± 501.66 |
| group according to | | | | | |
| the present invention | | | | | |

### 8-1-3. Confirmation of Amount of IFN-γ Secretion

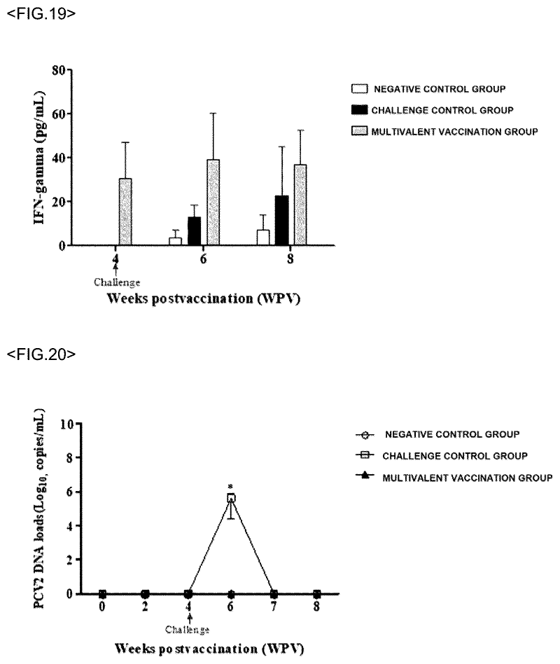

To confirm the amount of IFN-γ secretion, PBMCs were isolated and stimulated with the antigen. Ficoll was dispensed by 4 mL into each conical tube and prepared. 4 mL of blood and 4 mL of PBS from each experimental group were mixed and prepared in a new conical tube. The blood and PBS mixture was slowly transferred to a tube containing Ficoll and then centrifuged at room temperature for 30 minutes at a speed of 840×g. PBMCs floating between Ficoll and serum were isolated using a pipette and then transferred to a new conical tube containing 4 mL of PBS. The PBMCs were centrifuged at room temperature at a speed of 210×g for 10 minutes. After removing the supernatant, the PBMCs were resuspended in 3 mL of PBS and then centrifuged at room temperature for 10 minutes at a speed of 210×g. The supernatant was removed, resuspended in PBS in the same manner, and centrifuged to remove the supernatant. Then, the cells were suspended by adding 1 mL of RPMI (10% FBS, 1% Anti-anti). 10 μL of PBMCs were mixed with 1% trypan blue solution at 1:1, counted with a cell counter, and then dispensed at 1×10⁶ cells/well in a 96-well plate. After 30 minutes of PBMC dispensing, 100 μL/well each of recombinant PCV2d protein purified antigen (in RPMI) was dispensed and stimulated at a concentration of 10 μg/mL, cultured for 72 hours, and then centrifuging at a speed of 15,800×g for 10 minutes to isolated the supernatant. The isolated supernatant was recovered and stored at −70° C. For the supernatant, the amount of IFN-γ secretion was measured using an IFN-γ Porcine ELISA kit (Invitrogen, #KSC4021) according to the manufacturer's instructions. The results of measuring the amount of IFN-γ secretion were shown in FIG. 19 and Table 9 below.

As a result, at 4WPV, in the multivalent vaccination group according to the present invention, 30.62 pg/mL of PCV2d antigen-specific IFN-γ was secreted, and there was no significant difference in all experimental periods. On the other hand, IFN-γ was not measured in the challenge control group and the negative control group.

TABLE 9

| | IFN-γ average secretion amount of | | |
|---|---|---|---|
| | PCV2d sensitization group | | |
| Experimental | after challenge (pg/mL, AVE ± SE) | | |
| group name | 4 WPV | 6 WPV | 8 WPV |
| Negative control | 0.00 ± 0.00 | 3.60 ± 3.60 | 7.25 ± 7.25 |
| group | | | |
| Challenge control | 0.00 ± 0.00 | 12.80 ± 5.85 | 22.80 ± 22.25 |
| group | | | |
| Multivalent | 30.62 ± 16.54 | 39.26 ± 21.19 | 36.84 ± 15.86 |
| vaccination group | | | |
| according to the | | | |
| present invention | | | |

### 8-2. Quantification of Challenge Virus DNA

The challenge virus DNA was quantitatively evaluated in serum, nasal specimen (swab), rectal specimen (swab), lung, and lymph nodes of each experimental group. First, the serum was isolated from the blood of all subjects in each group, and nasal and rectal samples were collected using cotton swabs. Each sample was placed in a 2 mL E-tube and thoroughly vortexed with 1 mL of PBS to mix the sample and PBS. At this time, the lung and lymph node tissues were mixed with PBS in a 2 mL round-bottom E-tube and lysed using TissueLyser II with beads.

The mixture was centrifuged at 10,000×g for 5 minutes to isolate the supernatant. Thereafter, viral DNA was isolated using Gene-spin™ Viral DNA/RNA Extraction Kit. For PCV2 quantitative analysis, RT-qPCR was performed using primers for PCV2 (forward primer (5′-AACCACATACTGGAAACCAC-3′ (SEQ ID NO: 5)) and reverse primer (5′-TTGAGGAGTACCATTCCAAC-3′ (SEQ ID NO: 6)).

The reagent composition for PCV2 detection was shown in Table 10, and RT-qPCR was performed according to the conditions in Table 11.

TABLE 10

| Reagent | Volume (μL) |
|---|---|
| TOPreal ™ qPCR 2X PreMIX | 10 |
| Viral DNA template | 5 |
| 10 pM Forward primer | 1 |
| 10 pM Reverse primer | 1 |
| Sterile DDW | 3 |

TABLE 11

| PCR conditions | Temperature (° C.) | Time | | cycle |
|---|---|---|---|---|
| Initial denaturation | 95 | 10 | min | 1 |
| Denaturation | 95 | 10 | sec | 35 |
| Annealing | 53 | 15 | sec | |
| Elongation | 72 | 15 | sec | |
| Melt Curve stage | 95 | 15 | sec | 1 |
| | 60 | 1 | min | 1 |
| | 80~95 | 15 sec/1 step | | 1 |
| | 0.3° C./1 step | | | |

When performing RT-qPCR, a negative control group and a standard sample (10³ to 10⁹ DNA copies) for PCV2d were always performed together. After RT-qPCR, the number of DNA copies of the sample was measured by substituting the standard quantitative curve obtained from the standard sample. At this time, no amplification products should be observed in the negative control group. The results of the test samples were summarized and the presence or absence of PCV2d positive and negative was recorded.

### 8-2-1. Measurement Results of Serum, Nasal and Rectal Samples

The quantification results of PCV2 DNA copy number in serum (viremia) were shown in FIG. 20 and Table 12 below. In FIG. 20, * represented a result compared with the negative control group (P<0.05).

As a result, PCV2 DNA was not detected in the serum of all experimental groups from after vaccination to before challenge (4 WPV). In addition, even after challenge, viremia was not observed in the multivalent vaccination group according to the present invention, like the negative control group. However, the challenge control group showed viremia of 5.63 (Log₁₀) copies/mL at 2 weeks after challenge (6 WPV). From 7 WPV to 8 WPV, viremia was not observed in all experimental groups.

TABLE 12

| | PCV2 DNA load (Log₁₀, copies/mL, AVE ± SE) | | | | | |
|---|---|---|---|---|---|---|
| Group name | 0 WPV | 2 WPV | 4 WPV | 6 WPV | 7 WPV | 8 WPV |
| Negative control group | 0.00 ± 0.00 | 0.00 ± 0.00 | 0.00 ± 0.00 | 0.00 ± 0.00 | 0.00 ± 0.00 | 0.00 ± 0.00 |
| Challenge control group | 0.00 ± 0.00 | 0.00 ± 0.00 | 0.00 ± 0.00 | 5.63 ± 0.53 | 0.00 ± 0.00 | 0.00 ± 0.00 |
| Multivalent vaccination | 0.00 ± 0.00 | 0.00 ± 0.00 | 0.00 ± 0.00 | 0.00 ± 0.00 | 0.00 ± 0.00 | 0.00 ± 0.00 |
| group according to | | | | | | |
| the present invention | | | | | | |

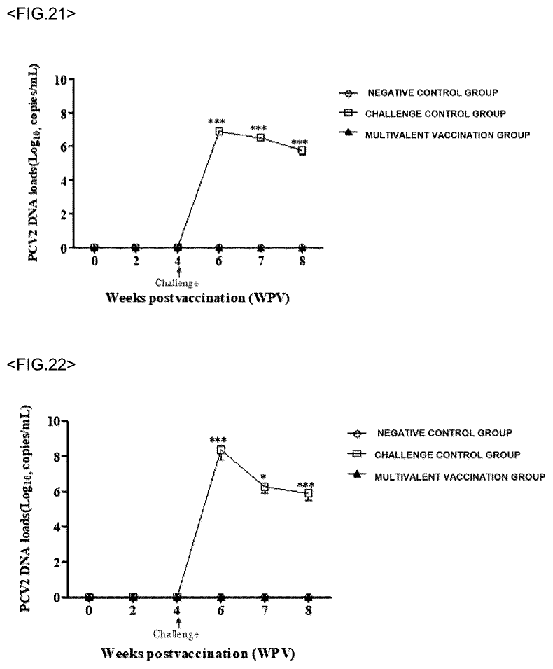

The results of quantifying PCV2 in the nasal cavity and rectum were shown in FIGS. 21 and 22 and Tables 13 and 14 below. In FIG. 21, *** represented a result compared with the negative control group (P<0.001). In addition, in FIG. 22, * represented a result compared with the negative control group (P<0.05), and *** represented a result compared with the negative control group (P<0.001). In addition, Table 13 shows the results of quantifying the PCV2 DNA copy number in the nasal cavity, and Table 14 shows the results of quantifying the PCV2 DNA copy number in the rectum.

As a result, PCV2 DNA was not detected in the serum of all experimental groups from after vaccination to before challenge (4 WPV). From 6 WPV, PCV2 DNA was detected only in the challenge control group, and significantly higher levels of DNA were detected at 6.88 (Log₁₀) and 8.36 (Log₁₀) copies/mL in the nasal cavity and rectum, respectively, which showed a significant difference compared to other groups, and DNA tended to gradually decrease until 8 WPV.

TABLE 13

| Experimental | PCV2 DNA load (Log₁₀, copies/mL, AVE ± SE) | | | | | |
|---|---|---|---|---|---|---|
| group name | 0 WPV | 2 WPV | 4 WPV | 6 WPV | 7 WPV | 8 WPV |
| Negative control group | 0.00 ± 0.00 | 0.00 ± 0.00 | 0.00 ± 0.00 | 0.00 ± 0.00 | 0.00 ± 0.00 | 0.00 ± 0.00 |
| Challenge control group | 0.00 ± 0.00 | 0.00 ± 0.00 | 0.00 ± 0.00 | 6.88 ± 0.59 | 6.51 ± 0.54 | 5.77 ± 0.51 |
| Multivalent vaccination | 0.00 ± 0.00 | 0.00 ± 0.00 | 0.00 ± 0.00 | 0.00 ± 0.00 | 0.00 ± 0.00 | 0.00 ± 0.00 |
| group according to | | | | | | |
| the present invention | | | | | | |

TABLE 14

| Experimental | PCV2 DNA load (Log₁₀, copies/mL, AVE ± SE) | | | | | |
|---|---|---|---|---|---|---|
| group name | 0 WPV | 2 WPV | 4 WPV | 6 WPV | 7 WPV | 8 WPV |
| Negative control group | 0.00 ± 0.00 | 0.00 ± 0.00 | 0.00 ± 0.00 | 0.00 ± 0.00 | 0.00 ± 0.00 | 0.00 ± 0.00 |
| Challenge control group | 0.00 ± 0.00 | 0.00 ± 0.00 | 0.00 ± 0.00 | 8.36 ± 4.89 | 6.27 ± 3.62 | 5.89 ± 3.41 |
| Multivalent vaccination | 0.00 ± 0.00 | 0.00 ± 0.00 | 0.00 ± 0.00 | 0.00 ± 0.00 | 0.00 ± 0.00 | 0.00 ± 0.00 |
| group according to | | | | | | |
| the present invention | | | | | | |

### 8-2-2. Measurement Results of Lung and Lymph Node Samples

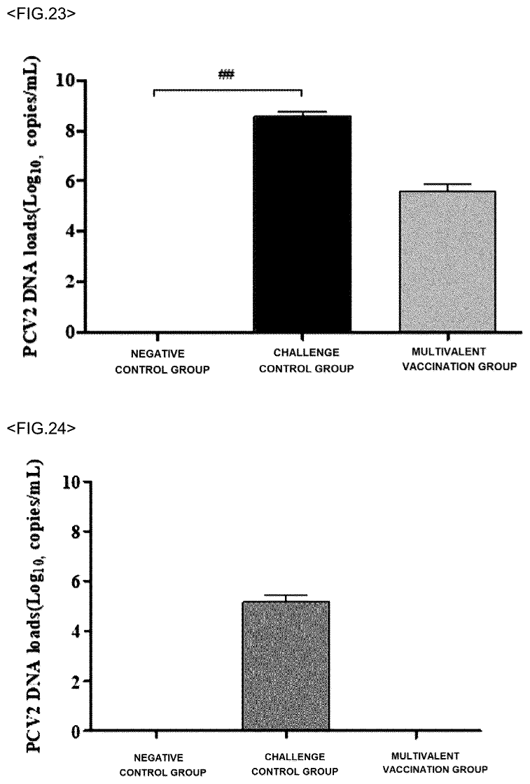

The results of examining the PCV2 DNA copy number in lung and inguinal lymph node tissues collected after euthanizing all experimental groups at 8 WPV, the end of the test, were shown in FIGS. 23 and 24 and Tables 15 and 16 below. In FIG. 23, ##represented a result compared with the challenge control group (P<0.01). In addition, Table 15 shows the results of quantifying the PCV2 DNA copy number in the lung, and Table 16 shows the results of quantifying the PCV2 DNA copy number in the lymph node.

As a result, the challenge control group showed a significantly higher level of DNA copy number in the lung at 8.56 (Log₁₀) copies/mL, but the multivalent vaccination group according to the present invention showed the DNA copy number of 5.59 (Log10) copies/mL, which was reduced by 1/10³ to be significantly lower than the challenge control group.

In the lymph nodes, the challenge control group showed the DNA copy number of 5.17 (Log₁₀) copies/mL, but the multivalent vaccination group showed no PCV2 DNA copy number. Meanwhile, in the negative control group, PCV2 DNA was not detected in the lung and lymph nodes.

TABLE 15

| | PCV2 DNA load (Log₁₀, copies/mL, |
|---|---|
| | AVE ± SE) |
| Experimental group name | 8 WPV |
| Negative control group | 0.00 ± 0.00 |
| Challenge control group | 8.56 ± 8.33 |
| Multivalent vaccination | 6.59 ± 5.54 |
| group according to | |
| the present invention | |

TABLE 16

| | PCV2 DNA load (Log₁₀, copies/mL, |
|---|---|
| | AVE ± SE) |
| Experimental group name | 8 WPV |
| Negative control group | 0.00 ± 0.00 |
| Challenge control group | 5.17 ± 5.17 |
| Multivalent vaccination | 0.00 ± 0.00 |
| group according to | |
| the present invention | |

### 8-3. Pathological Evaluation

### 8-3-1. Visual Evaluation

Autopsy and visual lesion evaluation were conducted by requesting Optipharm Co., Ltd., and the results were shown in Tables 17 and 18 below. The results of visual lesion evaluation were shown in Table 17, and the results of histological evaluation of the inguinal lymph nodes were shown in Table 18.

As a result of visual observation, changes were confirmed in the challenge control group among four experimental groups. Superficial inguinal lymph nodes did not show a significant difference in size between the experimental groups, but when measuring the size of the lymph nodes, the lymph nodes of subjects 1 and 2 of the challenge control group were slightly larger than those of the other experimental groups. In the lungs, purplish-red pulmonary consolidation was formed centered on the cranioventral lobe in the lung, and pulmonary consolidation was confirmed only in Subjects 1 and 2 of the challenge control group. In addition, in Subject 2 of the challenge control group, pericardial adhesion was observed on the surface of the heart due to an inflammatory exudate, presumed to be milky-white fibrin. No significant visual changes were observed in other organs.

TABLE 17

| | | Organ | | |
|---|---|---|---|---|
| Experimental | Subject | Inguinal | | |
| group name | No. | lymph nodes | Lung | Heart |
| Negative | 1 | — | — | — |
| control group | 2 | — | — | — |
| Challenge | 1 | Enlarged | Purplish-red | — |
| control group | | lymph nodes | pulmonary | |
| | | | consolidation | |
| | 2 | Enlarged | Purplish-red | Fibrous |
| | | lymph nodes | pulmonary | pericardial |
| | | | consolidation | adhesion |
| | 3 | — | — | — |
| Multivalent | 1 | — | — | — |
| vaccination group | 2 | — | — | — |
| according to | 3 | — | — | — |
| the present | 4 | — | — | — |
| invention | 5 | — | — | — |

TABLE 18

| Experimental | Subject | Left (cm) | | Right (cm) | |
|---|---|---|---|---|---|
| group name | No. | Horizon | Vertical | Horizon | Vertical |
| Negative | 1 | 1.0 | 3.0 | 1.1 | 2.8 |
| control group | 2 | 1.0 | 2.5 | 1.0 | 2.5 |
| Challenge | 1 | 1.4 | 3.0 | 1.4 | 3.0 |
| control group | 2 | 1.4 | 3.2 | 1.2 | 3.2 |
| | 3 | 1.2 | 2.5 | 1.2 | 2.8 |
| Multivalent | 1 | 1.3 | 2.7 | 1.2 | 3.5 |
| vaccination group | 2 | 1.0 | 2.5 | 1.1 | 2.4 |
| according to | 3 | 1.4 | 2.5 | 1.4 | 2.5 |
| the present invention | 4 | 1.3 | 2.4 | 1.2 | 2.3 |
| | 5 | 1.5 | 2.4 | 1.2 | 2.0 |

### 8-3-2. Histopathological and Immunohistochemical Evaluation

After visual observation of the internal organs of each experimental group, major internal organs were extracted and some thereof were fixed in a 10% neutral buffered formalin (NBF) solution.

The fixed tissues were trimmed to observe the cross-section of each organ, embedded in paraffin according to a general tissue processing method, and sliced to a thickness of about 3 to 4 μm to produce tissue slides. The slides were stained with hematoxylin & eosin (H&E) and then examined using an optical microscope (Olympus BX53, Japan).

Additional tissue sections of the lung, lymph nodes, and tonsils were subjected to immunohistochemistry (IHC) using a specific antibody against PCV2 to confirm the presence of porcine circovirus type 2 (PCV2) in each organ. The IHC analysis was conducted by requesting Optipharm Co., Ltd. The results thereof were shown in FIG. 19 and Table 20 below. Table 19 shows results of histopathological and immunohistochemical evaluation of lymph nodes, lungs, and kidneys, and Table 20 shows results of histopathological and immunohistochemical evaluation of organs.

As a result of histopathological test, lymphocyte depletion and histiocytic replacement were observed in some lymph nodes. The findings were characterized by a decrease in the number of lymphocytes due to degeneration and necrosis of lymphocytes within lymphatic follicles, and infiltration of cells such as histiocytes in the corresponding space as part of an inflammatory response. The finding is one of the histopathological findings that may be diagnosed as porcine circovirus-related disease (PCVAD) in piglets after weaning, and PCVAD may be diagnosed when viral antigens are detected in the cells constituting the lesion, such as infiltrated histiocytes. In the lungs, perivascular and peribronchiolar cuffing, BALT hyperplasia, suppurative bronchointerstitial pneumonia, peribronchiolar fibroplasia with bronchiolar segmentation and pulmonary edema were mainly confirmed. Among them, the perivascular and peribronchiolar cuffing is a change that occurs early in common respiratory infections, including PCV2, and the pulmonary edema may also be interpreted as part of the early inflammatory response. Characteristically, the peribronchiolar fibroplasia is a finding in which cells constituting connective tissue, such as fibroblasts, increase around the peribronchioles. As the increase in connective tissue becomes more severe, the lumen of the bronchioles was pressed, leading to segmentation or obstruction of the airway lumen. Since these findings are more frequently observed in PCVAD, the findings were known to be one of the changes that may suspect PCV2 infection. The above-mentioned lesions such as inflammatory changes and edema around the airway become more severe to progress to pneumonia, and in Example, suppurative bronchointerstitial pneumonia was confirmed in the consolidation of the cranioventral lobe. The finding was judged to be the result of coinfection of secondary respiratory bacteria along with respiratory viruses. Additionally, the BALT hyperplasia is a finding formed by chronic antigen stimulation of the airway epithelial layer after infection. When observed alone without pneumonia or other inflammatory changes, it is usually judged to be changes caused by respiratory mycoplasma infection. In addition, in the kidney, interstitial inflammatory cell infiltration was observed mainly in the cortical area. Most of the interstitially infiltrated inflammatory cells were mononuclear inflammatory cells, which formed non-suppurative inflammation and were known to be changes caused by viral infections, including PCV2. As a result of comparing the appearance of histopathological lesions by group, lymph node findings were observed only in the challenge control group. The lymphoid depletion and histiocytic replacement were observed at a slight level only in Subject 1 of the challenge control group, and lymph node lesions judged as PCV2 infection were not significant in all experimental subjects. In the case of lung lesions, mild or less perivascular and peribronchiolar cuffing was observed in all subjects in the multivalent vaccination group according to the present invention. In addition, mild or less BALT hyperplasia was confirmed in Subjects 2 and 3 of the multivalent vaccination group according to the present invention. In the case of the challenge control group, moderate or higher perivascular and peribronchiolar cuffing was confirmed in all subjects. Subject 1 of the challenge control group developed moderate or higher suppurative bronchointerstitial pneumonia centered on the cranioventral lobe, accompanied by mild pulmonary edema in some alveoli, showing the most severe findings among all subjects. Subject 2 had severe infiltration of inflammatory cells around the airways, accompanied by mild peribronchiolar fibroplasia, and some of the bronchiolar lumens were deformed and segmented. Moderate pulmonary edema was also confirmed in the alveoli. Subject 3 showed only moderate perivascular and peribronchiolar cuffing and slight BALT hyperplasia, showing the mildest changes among the three subjects in the challenge control group. In the negative control group, only mild or less perivascular and peribronchiolar cuffing was observed in both the subjects. In the kidney, moderate or lower interstitial inflammatory cell infiltration was locally or multifocally observed only in the challenge control group. The lesion was most prominent at a moderate level in Subject 1 of the challenge control group.

In addition, as a result of IHC of PCV2 in superficial inguinal lymph nodes, lungs, and kidneys, PCV2 antigen was not detected in all organs in the multivalent vaccination group according to the present invention. In the challenge control group, a positive reaction limited to a few cells was confirmed in lymph nodes of Subjects 1 and 2, and a mild positive reaction was confirmed in some cells infiltrated in the lesion in the lung 1. In the kidney tissue, positive reactions were confirmed in all subjects in the challenge control group, and most of some cells constituting interstitial inflammatory lesions showed positive reactions. The positive reaction was observed at a mild level in kidney 1, and at a weak level limited to the local area in the remaining subjects. In the negative control group, the PCV2 antigen was not detected in all organs.

TABLE 19

| | | Group name | | |
|---|---|---|---|---|
| | | | | Multivalent |
| | | | | vaccination |
| | | | | group |
| | | Negative | Challenge | according to |
| | | control | control | the present |
| Organ | Symptoms | group | group | invention |
| Inguinal | Lymphoid depletion and | 0/2* | 1/3 | 0/5 |
| lymph | histiocytic replacement | | | |
| nodes | of lymph follicles | | | |
| | PCV2 Detection | 0/2 | 2/3 | 0/5 |
| Lung | Perivascular and | 2/2 | 3/3 | 5/5 |
| | peribronchiolar cuffing | | | |
| | BALT hyperplasia | 0/2 | 1/3 | 2/5 |
| | Suppurative | 0/2 | 1/3 | 0/5 |
| | bronchointerstitial | | | |
| | pneumonia | | | |
| | Peribronchiolar | 0/2 | 1/3 | 0/5 |
| | fibroplasia | | | |
| | with bronchiolar | | | |
| | segmentation | | | |
| | Pulmonary edema | 0/2 | 2/3 | 0/5 |
| | PCV2 Detection | 0/2 | 1/3 | 0/5 |
| Kidney | Interstitial inflammatory | 0/2 | 3/3 | 0/5 |
| | cell infiltration | | | |
| | PCV2 Detection | 0/2 | 3/3 | 0/5 |

*Number of detected subjects/Total number of subjects

TABLE 20

| | Number | | Symptoms | |
|---|---|---|---|---|
| | of | | | PCV2 antigen |
| Group name | subject | Organ | Histopathological lesions ¹⁾ | detection²⁾ |
| Negative | 1 | Inguinal | — | − |
| control group | | lymph node | | |
| | | Lung | Perivascular and peribronchiolar cuffing (1+) | − |
| | | Kidney | — | − |
| | 2 | Inguinal | — | − |
| | | lymph node | | |
| | | Lung | Perivascular and peribronchiolar cuffing (2+) | − |
| | | Kidney | — | − |
| Challenge | 1 | Inguinal | Lymphoid depletion and histiocytic replacement | + |
| control group | | lymph node | of lymph follicles (1+) | |
| | | Lung | Perivascular and peribronchiolar cuffing (3+) | ++ |
| | | | Suppurative bronchointerstitial pneumonia (3+) | |
| | | | Pulmonary edema (1+) | |
| | | Kidney | Interstitial inflammatory cell infiltration (3+) | ++ |
| | 2 | Inguinal | — | + |
| | | lymph node | | |
| | | Lung | Perivascular and peribronchiolar cuffing (4+) | − |
| | | | Peribronchiolar fibroplasia with | |
| | | | bronchiolar segmentation (2+) | |
| | | | Pulmonary edema (3+) | |
| | | Kidney | Interstitial inflammatory cell infiltration (1+) | + |
| | 3 | Inguinal | — | − |
| | | lymph node | | |
| | | Lung | Perivascular and peribronchiolar cuffing (3+) | − |
| | | | lymphoid tissue hyperplasia (1+) | |
| | | Kidney | Interstitial inflammatory cell infiltration (2+) | + |
| Multivalent | 1 | Inguinal | — | − |
| vaccination group | | lymph node | | |
| according to | | Lung | Perivascular and peribronchiolar cuffing (1+) | − |
| the present invention | | Kidney | — | − |
| | 2 | Inguinal | — | − |
| | | lymph node | | |
| | | Lung | Perivascular and peribronchiolar cuffing (2+) | − |
| | | Kidney | — | − |
| | 3 | Inguinal | — | − |
| | | lymph node | | |
| | | Lung | Perivascular and peribronchiolar cuffing (2+) | − |
| | | | lymphoid tissue hyperplasia (2+) | |
| | | Kidney | — | − |
| | 4 | Inguinal | — | − |
| | | lymph node | | |
| | | Lung | Perivascular and peribronchiolar cuffing (2+) | − |
| | | | lymphoid tissue hyperplasia (2+) | |
| | | Kidney | — | − |
| | 5 | Inguinal | — | − |
| | | lymph node | | |
| | | Lung | Perivascular and peribronchiolar cuffing (2+) | − |
| | | Kidney | — | − |

¹⁾ Histopathological findings: (1+) Minimum; (2+) Mild; (3+) Moderate; (4+) Severe.
²⁾Immunohistochemical findings: (−) None; (+) Low; (++) Medium; (++++) High.

Comprehensively, the multivalent vaccine composition of Mhp+Mhr+recombinant P97 protein+recombinant PCV2 capsid antigen combination according to the present invention exhibited a reciprocally supplemental effect on immune responses when inoculated into mice and guinea pigs. Particularly, when inoculated into pigs used as target animals, the multivalent vaccine composition induced high serological levels of antibody titers and neutralizing antibody titers and produced a high level of IFN-γ for Mhp or PCV2 stimulation in PBMC, and when inoculated into pigs used as target animals and challenged, the multivalent vaccine composition showed excellent defense ability without side effects. Thus, the multivalent vaccine composition may find advantageous applications for preventing porcine mycoplasma and porcine circovirus infections.

1. A multivalent vaccine composition for prevention of porcine mycoplasma and porcine circovirus infections comprising:
(i) a porcine Mycoplasma hyopneumoniae (Mhp) strain;
(ii) a porcine Mycoplasma hyorhinis (Mhr) strain;
(iii) a porcine Mycoplasma hyopneumoniae-derived recombinant P97 protein; and
(iv) a porcine circovirus type 2 (PCV2)-derived recombinant protein.

2. The multivalent vaccine composition of claim 1, wherein the recombinant P97 protein consists of an amino acid sequence represented by SEQ ID NO: 1.

3. The multivalent vaccine composition of claim 1, wherein the PCV2-derived recombinant protein consists of an amino acid sequence represented by SEQ ID NO: 2.

4. The multivalent vaccine composition of claim 1, wherein the porcine Mycoplasma hyopneumoniae is included at a concentration of 1×10⁸ to 1×10⁹ colour-changing units (CCU)/mL.

5. The multivalent vaccine composition of claim 1, wherein the porcine Mycoplasma hyorhinis is included at a concentration of 1×10⁸ to 1×10⁹ CCU/mL.

6. The multivalent vaccine composition of claim 1, wherein the porcine Mycoplasma hyopneumoniae-derived recombinant P97 protein is included at a concentration of 10 to 100 μg/dose.

7. The multivalent vaccine composition of claim 1, wherein the PCV2-derived recombinant protein is included at a concentration of 0.1 to 1000 μg/dose.

8. The multivalent vaccine composition of claim 1, wherein the porcine Mycoplasma hyopneumoniae strain is inactivated.

9. The multivalent vaccine composition of claim 1, wherein the porcine Mycoplasma hyorhinis strain is inactivated.

10. The multivalent vaccine composition of claim 1, wherein the multivalent vaccine composition further comprises an excipient.

11. The multivalent vaccine composition of claim 10, wherein the excipient is IMS1313.

12. A method for preventing porcine mycoplasma and porcine circovirus infections comprising inoculating the multivalent vaccine composition of claim 1 into pigs.

13. The prevention method of claim 12, wherein the multivalent vaccine composition is inoculated by one or more methods selected from the group consisting of intramuscular, subcutaneous, transdermal, intravenous, intranasal, intraperitoneal, and oral routes.

14. A pharmaceutical composition for preventing diseases caused by porcine mycoplasma and porcine circovirus infections comprising:
(i) a porcine Mycoplasma hyopneumoniae (Mhp) strain;
(ii) a porcine Mycoplasma hyorhinis (Mhr) strain;
(iii) a porcine Mycoplasma hyopneumoniae-derived recombinant P97 protein; and
(iv) a porcine circovirus type 2 (PCV2)-derived recombinant protein.

15. The pharmaceutical composition of claim 14, wherein the diseases caused by porcine mycoplasma and porcine circovirus infections are one or more selected from the group consisting of porcine respiratory disease complex (PRDC), enzootic pneumonia (EP), postweaning multisystemic wasting syndrome (PMWS), porcine dermatitis and nephropathy syndrome (PDNS), sow abortion and mortality syndrome (SAMS), porcine reproductive and respiratory syndrome (PRRS), pseudorabies, Glasser's disease, streptococcal meningitis, salmonellosis, postweaning colibacillosis, dietetic hepatosis, suppurative bronchopneumonia, Eustachian tube inflammation, polyserositis, mycoplasma pneumonia and pleurisy pneumonia.
